(19) United States
(12) Patent Application Publication
(10) Pub. No.: US 2026/0079169 A1
(43) Pub. Date: Mar. 19, 2026

(54) VOLATILE ORGANIC COMPOUNDS AS MARKERS FOR CELLULAR COMMUNICATION

(71) Applicant: Technion Research and Development Foundation Limited, Haifa (IL)

(72) Inventors: Hossam HAICK, Haifa (IL); Yoav BROZA, Haifa (IL); Mamatha SERASANAMBATI, Saint Louis, MO (US); Dina HASHOUL, Haifa (IL); Walaa SALIBA, Shefar'am (IL)

(21) Appl. No.: 19/398,697

(22) Filed: Nov. 24, 2025

Related U.S. Application Data
(63) Continuation of application No. 17/183,511, filed on Feb. 24, 2021.
(60) Provisional application No. 62/981,540, filed on Feb. 26, 2020.

(51) Int. Cl. G01N 33/84; C12N 5/07; G01N 33/574

(52) U.S. Cl. CPC G01N 33/84; C12N 5/06; G01N 33/5752; G01N 2496/00

(57) ABSTRACT

A method that evaluates metastatic behavior, apoptotic behavior, and/or cancer therapy response efficiency. The method has steps. The method has steps and some of those steps are: Providing a first cell culture having cancer cells and a second cell culture. There is no physical contact between the first cell culture and the second cell culture. Co-culturing the first cell culture and the second cell culture having no physical contact therebetween under a mutual headspace. Determining concentrations of VOCs in the mutual headspace. Comparing the concentrations of the VOCs in the mutual headspace to the concentrations of VOCs in a control sample. Identifying a set of VOCs indicative of cell-to-cell signaling in cancer cells in the mutual headspace wherein the VOCs have concentrations that are significantly different as compared to the control sample. And evaluating metastatic behavior, apoptotic behavior, and/or cancer therapy response efficiency using the set of VOCs identified above.

### CROSS REFERENCE TO RELATED APPLICATIONS

The present invention claims priority to U.S. Provisional Patent Application, Ser. No. 62/981,540, filed on Feb. 26, 2020, which is incorporated by reference herein in its entirety.

### FIELD OF THE INVENTION

The present invention relates to methods of detecting cell-to-cell signaling in cancer cells and diagnosing or monitoring cancer progression using volatile organic compounds indicative of cell-to-cell signaling, and a method of identifying such set of volatile organic compounds.

### BACKGROUND OF THE INVENTION

Studying cell-to-cell cross-talk in a group of cells is indispensable and provides a rich source of information. It is particularly important in cancer research since it has a major role in cancer development, tumorigenesis, metastasis and cell apoptosis. Major key factors in these communication networks are the cell-surface receptors, which are responsible for the transduction of signals through branched cascade of responses.

Cancer cells and tumor microenvironment (TME) are known to communicate through numerous diverse chemical mechanisms. This includes autocrine signaling that is expressed via secreting chemical messenger that binds to autocrine receptors on the same cell, which in turn affects the way the cell functions; and paracrine signaling, which is a form of signaling in which one cell affects neighboring ones by secreting chemicals into the common intercellular space. In addition, cells can directly transfer ions or small molecules from one cell to another through pores in the cell membrane. Several studies have shown that cell-to-cell cross-talk and tumorigenesis is highly influenced by specific stromal factors and TME components (Allinen, M. et al. Molecular characterization of the tumor microenvironment in breast cancer. Cancer cell. 6, 17 (2004)).

Various cell-to-cell communication mechanisms (e.g., cell proliferation, migration, cell recognition and differentiation) have been reported by proteomics and genomics approaches. Though tremendous advances have been achieved with these approaches, several limitations still restrict the fulfilment of the approaches in diagnostic or therapeutic applications. These limitations include, but not are confined to: 1) proteomics and genomics require prior and quite accurate knowledge of specific genes or proteins and are exclusive to in-vitro and in-vivo trials, which does not necessarily reflect real-life situations; and 2) genomics and proteomics still suffer from high cost, low specificity and complex analysis algorithms, resulting in prolonged and cumbersome analysis. Additionally, cancer is a systematic disease (polygenetic) involving various mutations on several levels (including, inter alia, genetic, epigenetic, in local or at distant to the primary tumor). Thus, a more comprehensive observation and broad analysis is of interest.

Volatile organic compounds (VOCs) are chemical compounds that have a low molecular weight and relatively high vapor pressure under room temperature conditions. Exhaled VOCs have captured an increased interest in recent years because of their potential role in the diagnosis of various types of diseases. Disease-specific VOCs can be used as diagnostic olfactory biomarkers of infectious diseases, metabolic diseases, genetic disorders and other kinds of diseases, including cancer. VOCs can be detected from samples of bodily fluids or the headspace of a container containing infected cells and/or tissues or directly from exhaled breath in which disease-related changes are reflected through exchange via the blood or directly via the lung airways.

In exhaled breath of patients with cancer, elevated levels of certain VOCs including volatile C₄-C₂₀ alkane compounds, specific monomethylated alkanes as well as benzene derivatives were found. Hence, the composition of VOCs in exhaled breath of patients with cancer differs from that of control individuals, and can therefore be used to diagnose cancer, and to monitor disease progression or therapy-mediated disease regression. In the past two decades many attempts have been made to identify one specific pattern of volatile organic compounds (VOCs) in the breath of lung cancer patients, involving the use of, inter alia, gas chromatography (GC), gas chromatography/mass spectrometry (GC-MS), electronic nose devices, capillary column GC and surface acoustic wave sensors (Phillips et al., Chest, 2003, 123, 2115-2123; Phillips et al., Cancer Biomarkers, 2007, 3, 95-109; Phillips et al.; Clinica Chimica Acta, 2008, 393, 76-84; Chen et al., Meas. Sci. Technol. 2005, 16, 1535-1546; Chen et al., Cancer, 2007, 110, 835-844; Di Natale et al., Biosensors and Bioelectronics, 2003, 18, 1209-1218; Gordon et al., Clin. Chem., 1985, 31 (8), 1278-1282; Song et al., Lung Cancer, 2009, 67, 227-231; O'neill et al., Clinical Chemistry, 1988, 34 (8), 1613-1617; Wehinger et al., Inter. J. Mass Spectrometry, 2007, 265, 49-59; Peng et al., Nature Nanotech, 2009, 4, 669-673; Schmidt K et al., J Biomark, 2015; 2015:981458. doi: 10.1155/2015/981458; Jia Z et al., ACS Omega, ACS Omega, 2018 3 (5), 5131-5140).

U.S. Pat. No. 8,597,953 to one of the inventors of the present invention is directed to a set of volatile organic compounds, comprising at least butylated hydroxy toluene or 4,6-di (1,1-dimethylethyl)-2-methyl-phenol for breath analysis, and to methods of use thereof in diagnosing, monitoring or prognosing lung cancer.

U.S. Pat. No. 9,315,848 to one the inventors of the present invention discloses methods of identifying a genetic abnormality such as mutation in EGFR or KRAS or ALK which is associated with the management of lung cancer or diagnosing, prognosing or monitoring the treatment of pre-cancerous conditions of the lung, such as bronchial dysplasia or atypical alveolar hyperplasia (AAH), through the detection of at least one volatile organic compound indicative of these states.

U.S. Pat. No. 9,528,979 to one of the inventors of the present application is directed to a unique profile of volatile organic compounds as breath biomarkers for lung cancer and provides diagnosis, prognosis and monitoring of lung cancer or predicting the response to an anti-cancer treatment through the detection of the unique profile of volatile organic compounds indicative of lung cancer at its various stages.

Experimental diagnostic methods involving VOCs sampling have also been directed to identifying other cancer types through breath (The Breast Journal, 2003, 9 (3), 184-191; Breast Cancer Research and Treatment, 2006, 99, 19-21; Metabolomics, 2007, 3 (1), 13-17; Head & Neck, 2008, 30 (6), 743-749; Schmidt K et al., J Biomark, 2015; 2015:981458; Jia Z et al., ACS Omega, ACS Omega, 2018 3 (5), 5131-5140).

U.S. Pat. No. 9,551,712 to one of the inventors of the present invention encompasses sets of VOCs for breath analysis, as well as, methods of use thereof in diagnosing, monitoring or prognosing breast cancer, head and neck cancer, prostate cancer or colon cancer.

In addition to the many studies that were aimed at identifying VOCs indicative of cancer from breath samples, several studies have been conducted to investigate the VOCs emitted to the cells' headspace from bulk (in vitro) cancer cell populations, which include millions of cells (Cancer Cell International, 2008, 8, 17; Anticancer Res., 29 (2009), pp. 419-426; Cancer 2007, 110:835-844; Nanomedicine (NBM) 2012; 8:580-589; Br. J. Cancer, 111 (2014), pp. 1213-2121; Int. J. Nanomedicine, 7 (2012), pp. 4135-4146; Nanomedicine, 9 (2013), pp. 758-766). In a recent study, Haick et al., have reported a VOC-based approach for single-cell analysis, utilizing lung cancer cells with various p53 genetic statuses and normal lung cells. The VOCs emitted to the cell's headspace were analyzed by gas chromatography combined with mass spectrometry. Among hundreds of detected compounds, 18 VOCs showed significant changes in their concentration levels in tumor cells versus control. The composition of these VOCs was found to depend, also, on the sub-molecular structure of the p53 genetic status (iScience, 11, 2019, 178-188).

The multiple studies of VOCs as cancer biomarkers, however, have failed to determine a single set of VOCs to be used as a diagnostic tool for screening of each type of cancer.

There is an unmet need for a new and unitary set of biomarkers that would provide a systematic evaluation of a proliferative disease without the need to isolate and explore specific genes or proteins. Preferably, such biomarkers would be capable of providing information on a single cell level, as well as large-scale screening. Determining a set of VOCs, which are indicative of cell signaling, would also be beneficial for providing cellular communication mapping of cancer tumors and assisting in the monitoring of metastatic behavior or response to therapy.

### SUMMARY OF THE INVENTION

The present invention provides a method of detecting cell-to-cell signaling in cancer cells by determining the levels of volatile organic compounds (VOCs), which are indicative of cell-to-cell signaling in a test sample. There is further provided a method of diagnosing, monitoring or prognosing cancer in a subject, which is based on the detection of said cell signaling VOCs biomarkers. A method of identifying a set of volatile organic compounds, which are indicative of cell-to-cell signaling in cancer cells, is further disclosed.

The present invention is based in part on the unexpected finding that cell-to-cell cross-talk in cancer cells can be detected by identifying specific volatile organic compounds (VOCs), which are chemical compounds that have a low molecular weight and relatively high vapor pressure at ordinary, room-temperature conditions. The inventors have shown that changes in nuclei and surface morphologies, as well as, the apoptotic behavior in lung cancer (LC) cells in co-cultured media having no physical contact therebetween were accompanied by the emission of VOCs at different stages of cancer cells growth. It has further been surprisingly discovered by the inventors that while some of the VOCs detected in the study were emitted directly by the cancer cells, other VOCs appeared in the co-cultured media headspace only. Without wishing to being bound by theory or mechanism of action, it is contemplated that said other VOCs were released as a result of inter-cellular communication. Additionally, some VOCs, which were produced by LC cells mono-cultures were not detected in the co-cultured media headspace. Without further wishing to being bound by theory or mechanism of action, it is assumed that said VOCs, which were emitted by cancer cells were consumed by the neighboring cells of the co-culture. Relative abundance of some VOCs, which were found both in the mono-culture and co-culture headspace, was different throughout the entire incubation period. The VOCs, which levels in the co-cultured media were significantly different as compared to their respective mono-cultures (including presence in mono-culture and absence in co-culture and vice versa) were elected as cancer cell signaling biomarkers (i.e., VOCs, which are indicative of cell-to-cell communication in cancer cells). Such biomarkers can be used not only for the detection of cellular communication, but also for diagnosing, prognosing and monitoring proliferative diseases and evaluating metastatic behavior and therapy response efficiency. The unique set of cell-to-cell signaling biomarkers, which were discovered by the inventors, were not previously recognized as being suitable for diagnosing lung cancer.

According to a first aspect, the present invention provides a method of detecting cell-to-cell signaling in cancer cells, the method comprising the steps of: collecting a test sample comprising volatile organic compounds (VOCs) from a test subject; identifying and determining the level of at least one VOC from the test sample, wherein the at least one VOC is indicative of cell-to-cell signaling in cancer cells; and comparing the level of the at least one VOC to a reference value.

The test sample can be selected from the group consisting of an isolated cell headspace, exhaled breath, skin volatiles, and bodily fluid or secretion of the test subject. Each possibility represents a separate embodiment of the invention.

According to some embodiments, the at least one VOC being indicative of cell-to-cell signaling is selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, and 4-methylbenzyl alcohol. According to further embodiments, the at least one VOC being indicative of cell-to-cell signaling is selected from the group consisting of 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol and 4-methylbenzyl alcohol. Each possibility represents a separate embodiment of the invention.

According to some embodiments, the cancer is lung cancer.

The reference value can be obtained from a database of levels of the at least one VOC measured from a co-culture headspace of a first cell culture comprising cancer cells and a second cell culture. According to some embodiments, the second cell culture comprises cells selected from the group consisting of normal cells, cancer cells which are identical to the cancer cells of the first cell culture, and cancer cells which are distinct from the cancer cells of the first cell culture.

According to some embodiments, an essentially similar level of the at least one VOC, which is indicative of cell-to-cell signaling in the test sample as compared to the reference value is indicative of cell-to cell signaling in cancer cells.

According to some embodiments, the method comprises identifying and determining the levels of a plurality of VOCs indicative of cell-to-cell signaling in cancer cells from the test sample which form a pattern and comparing the pattern to a plurality of reference values. The pattern can be analyzed with a pattern recognition analyzer comprising at least one algorithm selected from the group consisting of artificial neural network (ANN) algorithm, support vector machine (SVM), discriminant function analysis (DFA), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA). Each possibility represents a separate embodiment of the invention.

According to some embodiments, the step of identifying and determining the level of the at least one VOC from the test sample comprises the use of at least one technique selected from the group consisting of Gas-Chromatography (GC), GC-lined Mass-Spectrometry (GC-MS), Gas-Chromatography-Mass Spectrometry (GC-MS) combined with In-tube Extraction (ITEX), Proton Transfer Reaction Mass-Spectrometry (PTR-MS), Electronic nose device, and Quartz Crystal Microbalance (QCM). Each possibility represents a separate embodiment of the invention. In certain embodiments, the step of identifying and determining the level of the at least one VOC from the test sample comprises the use of Gas-Chromatography-Mass Spectrometry (GC-MS) combined with In-tube Extraction (ITEX).

According to some embodiments, the test subject is selected from a subject who is at risk of developing cancer, a subject who is suspected of having cancer, and a subject who is afflicted with cancer. Each possibility represents a separate embodiment of the invention.

According to another aspect, there is provided a method of diagnosing, monitoring or prognosing lung cancer in a subject comprising the steps of: collecting a test sample comprising volatile organic compounds (VOCs) from the subject; identifying and determining the level of at least one VOC from the test sample, wherein the at least one VOC is indicative of cell-to-cell signaling in lung cancer cells; and comparing the level of the at least one VOC to a reference value, wherein the reference value is obtained from a database of levels of the at least one VOC measured from a co-culture headspace of a first cell culture comprising lung cancer cells and a second cell culture, wherein the second cell culture comprises cells selected from the group consisting of normal cells, lung cancer cells which are identical to the lung cancer cells of the first cell culture and lung cancer cells which are distinct from the lung cancer cells of the first cell culture.

According to some embodiments, the test sample is selected from the group consisting of a headspace sample, exhaled breath, skin volatiles, and bodily fluid or secretion of the subject. Each possibility represents a separate embodiment of the invention.

According to some embodiments, the at least one VOC being indicative of cell-to-cell signaling is selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, and 4-methylbenzyl alcohol. According to further embodiments, the at least one VOC being indicative of cell-to-cell signaling is selected from the group consisting of 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol and 4-methylbenzyl alcohol. Each possibility represents a separate embodiment of the invention.

The reference value can be obtained from a database of levels of the at least one VOC measured from a co-culture headspace of a first cell culture comprising lung cancer cells and a second cell culture. According to some embodiments, the second cell culture comprises normal cells. According to additional embodiments, the second cell culture comprises lung cancer cells, which are identical to the lung cancer cells of the first cell culture. According to other embodiments, the second cell culture comprises lung cancer cells, which are distinct from the lung cancer cells of the first cell culture.

According to some embodiments, an essentially similar level of the at least one VOC, which is indicative of cell-to-cell signaling in the test sample as compared to the reference value is indicative of cancer or provides the prediction of the subject's response to a treatment regimen.

According to some embodiments, the method comprises identifying and determining the levels of a plurality of VOCs indicative of cell-to-cell signaling in cancer cells from the test sample which form a pattern and comparing the pattern to a plurality of reference values. The pattern can be analyzed with a pattern recognition analyzer comprising at least one algorithm selected from the group consisting of artificial neural network (ANN) algorithm, support vector machine (SVM), discriminant function analysis (DFA), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PIS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA). Each possibility represents a separate embodiment of the invention.

According to some embodiments, the step of identifying and determining the level of the at least one VOC from the test sample comprises the use of at least one technique selected from the group consisting of Gas-Chromatography (GC), GC-lined Mass-Spectrometry (GC-MS), Gas-Chromatography-Mass Spectrometry (GC-MS) combined with In-tube Extraction (ITEX), Proton Transfer Reaction Mass-Spectrometry (PTR-MS), Electronic nose device, and Quartz Crystal Microbalance (QCM). Each possibility represents a separate embodiment of the invention. In certain embodiments, the step of identifying and determining the level of the at least one VOC from the test sample comprises the use of Gas-Chromatography-Mass Spectrometry (GC-MS) combined with In-tube Extraction (ITEX).

According to some embodiments, the subject is selected from a subject who is at risk of developing cancer, a subject who is suspected of having cancer, and a subject who is afflicted with cancer. Each possibility represents a separate embodiment of the invention.

According to some embodiments, the database of levels of the at least one VOC measured from the co-culture headspace consists essentially of VOCs, which levels measured from the co-culture headspace are significantly different than levels of said VOCs measured from each one of (I) a headspace of the first cell culture, (II) a headspace of the second cell culture, (III) a headspace of the first cell culture and the second cell culture, wherein there is a physical contact between the first cell culture and the second cell culture.

According to some embodiments, the method of diagnosing, monitoring or prognosing lung cancer in a subject further comprises a step of treating the test subject if cancer is diagnosed. In further embodiments, treating the test subject is performed by at least one of surgery, radiation therapy, chemotherapy, surveillance, adjuvant (additional) therapy, and targeted therapy, or a combination of these treatments.

According to another aspect, there is provided a method of identifying a set of volatile organic compounds (VOCs) indicative of cell-to-cell signaling in cancer cells, comprising the steps of: providing a first cell culture comprising cancer cells and a second cell culture, wherein there is no physical contact between the first cell culture and the second cell culture; co-culturing the first cell culture and the second cell culture under a mutual headspace; determining the concentrations of VOCs in the mutual headspace; comparing the concentrations of the VOCs in the mutual headspace to the concentrations of VOCs in a control sample; and identifying a set of VOCs in the mutual headspace having concentrations that are significantly different as compared to the control sample.

According to some embodiments, the control sample is selected from the group consisting of: (a) a headspace of the first cell culture, (b) a headspace of the second cell culture, (c) a headspace of the first cell culture and the second cell culture, wherein there is a physical contact between the first cell culture and the second cell culture, and any combination thereof. Each possibility represents a separate embodiment of the invention.

According to some embodiments, the second cell culture comprises cancer cells. In some related embodiments, the second cell culture comprises cancer cells, which are identical to the cancer cells of the first cell culture. In additional embodiments, the second cell culture comprises cancer cells, which are distinct from the cancer cells of the first cell culture. According to some embodiments, the cancer is lung cancer. According to some embodiments, the second cell culture comprises normal cells.

According to some embodiments, each one of the first cell culture and the second cell culture comprises at least about 1,000 cells.

According to some embodiments, the step of determining the concentrations of VOCs in the mutual headspace comprises the use of at least one technique selected from the group consisting of Gas-Chromatography (GC), GC-lined Mass-Spectrometry (GC-MS), Gas-Chromatography-Mass Spectrometry (GC-MS) combined with In-tube Extraction (ITEX), and Proton Transfer Reaction Mass-Spectrometry (PTR-MS). Each possibility represents a separate embodiment of the invention. In some exemplary embodiments, the step of determining the concentrations of VOCs in the mutual headspace comprises the use of Gas-Chromatography-Mass Spectrometry (GC-MS) combined with In-tube Extraction (ITEX). The ITEX can comprise the use of a microtrap filled with an adsorbent material, selected from the group consisting of Tenax TA, activated carbon, polydimethylsiloxane, and combinations thereof.

According to some embodiments, the concentration of the VOCs in the mutual headspace is determined within less than about 1 minute from the formation of a mutual headspace. Additionally or alternatively, the concentration of the VOCs in the mutual headspace can be determined after at least about 24 hours from the formation of a mutual headspace.

According to some embodiments, the VOCs in the mutual headspace form a pattern. In further embodiments, the step of comparing the concentrations of the VOCs in the mutual headspace to the concentrations of VOCs in a control sample comprises analyzing the pattern of the VOCs with a pattern recognition analyzer comprising at least one algorithm selected from the group consisting of artificial neural network (ANN) algorithm, support vector machine (SVM), discriminant function analysis (DFA), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, and canonical discriminant analysis (CDA). Each possibility represents a separate embodiment of the invention.

In some embodiments, the set of VOCs comprises at least one VOC selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, and 4-methylbenzyl alcohol.

According to another aspect, there is provided a set of volatile organic compounds (VOCs) as biomarkers of cell-to-cell signaling in cancer cells comprising at least one VOC selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, and 4-methylbenzyl alcohol. In certain embodiments, the set comprises at least one VOC selected from the group consisting of 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol and 4-methylbenzyl alcohol. In additional embodiments, the set of VOCs comprises at least five VOCs selected from the above list. In certain embodiments, the cancer is lung cancer.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1D: Schematic representation of the co-culture methodology, showing the volatile exchange between two cell types (where applicable), wherein FIG. 1A shows mono-cultures (Format-1); FIG. 1B shows same-cell pair co-culture headspace (Format-2); FIG. 1C shows different-cell pair co-culture headspace (Format-3); FIG. 1D shows different-cell pair co-culture having physical contact (Format-4). While Cell-A and Cell-B combinations are presented in this figure as an example, similarly other cell combinations were performed with Cell-C, Cell-D, etc.).

FIGS. 1E-1G: Cell morphological changes in lung cancer and normal cell lines at 24, 48 and 72 h, wherein FIG. 1E shows mono-culture groups (Format-1); FIG. 1F shows same-cell pair co-culture headspace (Format-2); and FIG. 1G shows different-cell pair co-culture headspace (Format-3). Cellular morphology was observed in three independent experiments (n=3). Photographs were taken directly from culture plates with a light inverted microscope (magnification ×40). Scale bar-50 μm

FIG. 2: Greyscale copies of ImageStream images of nuclear morphology of cells in Formats 1-4, stained with Hoechst 333442 dye, which stains morphologically normal nuclei dimly blue (shown as dimly grey), whereas apoptotic nuclei demonstrate reduced, intensely bright blue (shown as intense grey) and smaller nuclei.

FIGS. 3A-3E: Confluency of cells in Formats 1-4, wherein FIG. 3A shows mono-culture groups (Format-1) at 24, 48 and 72 h; FIG. 3B shows same-cell pair co-culture headspace (Format-2) at 24, 48 and 72 h; FIG. 3C shows different-cell pair co-culture headspace (Format-3) at 24 h; FIG. 3D shows different-cell pair co-culture headspace (Format-3) at 48 h; and FIG. 3E shows different-cell pair co-culture headspace (Format-3) at 72 h. Data presented as mean values ±SD of three experiments (p<0.05).

FIG. 4. Greyscale copy of a heat-map showing VOCs profile changes at different cell levels: 2, 4 and bulk cell levels, wherein A is BEAS-2B; B is A549; C is H1299; D is H1975, VOC1-VOC104 are indicated in Tables 5a-5b hereinbelow, color-coding shows the area under curve (AUC) of each compound measured at all cell levels, and the AUC values are normalized by STD AUC calculated for each sample.

### DETAILED DESCRIPTION OF THE INVENTION

The present invention provides a signature set of volatile organic compounds as cell-to-cell signaling biomarkers in cancer cells, which can be used not only for the detection of cellular communication, but also for diagnosing, prognosing and monitoring cancer proliferation. Further provided is a method of identifying said signature VOCs set.

The set of the VOCs described in the present invention comprises unique volatile organic compounds obtained from the headspace of co-cultured media of same or different types of lung cells, including cancer cells and normal cells, wherein there is no physical contact between said two types of cells. To the inventors' best knowledge, it was not previously realized that cell-to-cell signaling in cancer cells can be detected by measuring the levels of volatile organic compounds associated with said cells and that such specific VOCs may serve as biomarkers for cellular communication mapping of cancer tumors. Detection of cellular communication by means of identifying and measuring the levels of VOC biomarkers can be performed by simple, and highly accurate devices, such as, for example, GC-MS or electronic nose sensors, and painless and quick procedures, thus enabling efficient and inexpensive monitoring of metastatic behavior or cancer therapy response.

Throughout cell's life time, different metabolic processes occur to sustain life, and adjust to both systemic and local stimuli. When subjected to stress, cells typically response by triggering various intracellular cascades involving enzymes and signaling pathways, thereby adapting to changing micro-environmental requirements. Such pathophysiological processes occurring, inter alia, in the course of different diseases, can alter metabolism at the single cell level, as well as inducing entire organ response. At least some of these metabolites are VOCs, which might, therefore, have an important role in communication and disease metabolism. However, the effect of inter-specific interactions on VOC production and composition has been unknown up until now (Blom, D. et al., Environ. Microbio, 13, 3047 (2011); Garbeva, P. et al. Front. Microbiol. 5, 289 (2014)). The present invention, provides, for the first time, a method of detecting cell-to-cell singling in cancer cells based on the quantitative analysis of volatile organic compounds, which can be found, inter alia, in breath, various body fluids and in a cell culture or tissue headspace.

According to one aspect, the invention provides a method of detecting cell-to-cell signaling in cancer cells, the method comprising the steps of: (a) collecting a test sample comprising volatile organic compounds (VOCs) from a test subject; (b) identifying and determining the level of at least one VOC from the test sample, wherein the at least one VOC is indicative of cell-to-cell signaling in cancer cells; and (c) comparing the level of the at least one VOC to a reference value.

According to another aspect, there is provided a method of diagnosing, monitoring or prognosing cancer in a subject comprising the steps of: (a) collecting a test sample comprising volatile organic compounds (VOCs) from the subject; (b) identifying and determining the level of at least one VOC from the test sample, wherein the at least one VOC is indicative of cell-to-cell signaling in cancer cells; and comparing the level of the at least one VOC to a reference value.

In yet another aspect, there is provided a set of volatile organic compounds (VOCs) as biomarkers of cell-to-cell signaling in cancer cells.

The term “volatile organic compound”, as used herein, is meant to encompass semi-volatile organic compounds in addition to volatile organic compounds.

According to the various aspects and embodiments of the present invention, the at least one VOC being indicative of cell-to-cell signaling is a hydrocarbon or a ketone. According to further aspects and embodiments, the set of VOCs biomarkers of cell-to-cell signaling comprises at least one of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, 4-methylbenzyl alcohol, 2-fluoro-acetamide, ethanol, fumaronitrile, 1,3-bis(1,1-dimethylethyl)-benzene, 1,3-benzodioxole-2-carboxylic acid, ethyl ester, 5-(dimethylamino)-1,3-benzenedicarboxylic acid, and combinations thereof.

In some embodiments, the set of VOCS which are indicative of cell-to-cell signaling in cancer cells comprises at least two VOCs from the above list. In further embodiments, the set of VOCS which are indicative of cell-to-cell signaling in cancer cells comprises at least three VOCs from the above list, at least four VOCs, at least five VOCs, at least six VOCs, at least seven VOCs, at least eight VOCs, at least nine VOCs, or at least ten VOCs. Each possibility represents a separate embodiment of the invention.

In some embodiments, the method of detecting cell-to-cell signaling in cancer cells comprises (in step (b)) identifying and determining the level of at least two VOCs from the test sample, wherein the at least two VOCs are indicative of cell-to-cell signaling in cancer cells. In further embodiments, the method of detecting cell-to-cell signaling in cancer cells comprises (in step (b)) identifying and determining the level of at least three VOCs from the test sample, at least four VOCs, at least five VOCs, at least six VOCs, at least seven VOCs, at least eight VOCs, at least nine VOCs, or at least ten VOCs, wherein said VOCs are indicative of cell-to-cell signaling in cancer cells. In some related embodiments, the VOCs being indicative of cell-to-cell signaling in cancer cells are selected from the above list.

In some embodiments, the method of diagnosing, monitoring or prognosing cancer in a subject comprises (in step (b)) identifying and determining the level of at least two VOCs from the test sample, wherein the at least two VOCs are indicative of cell-to-cell signaling in cancer cells. In further embodiments, the method of detecting cell-to-cell signaling in cancer cells comprises (in step (b)) identifying and determining the level of at least three VOCs from the test sample, at least four VOCs, at least five VOCs, at least six VOCs, at least seven VOCs, at least eight VOCs, at least nine VOCs, or at least ten VOCs, wherein said VOCs are indicative of cell-to-cell signaling in cancer cells. In some related embodiments, the VOCs being indicative of cell-to-cell signaling in cancer cells are selected from the above list.

According to certain embodiments, the VOCs, which are indicative of cell-to-cell signaling in cancer cells are selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, and 4-methylbenzyl alcohol, and combinations thereof. In certain embodiments, the VOCs, which are indicative of cell-to-cell signaling in cancer cells are selected from the group consisting of 2-fluoro-acetamide, ethanol, fumaronitrile, 1,3-bis(1,1-dimethylethyl)-benzene, 1,3-benzodioxole-2-carboxylic acid, ethyl ester, 5-(dimethylamino)-1,3-benzenedicarboxylic acid, and combinations thereof. In certain embodiments, the VOCs, which are indicative of cell-to-cell signaling in cancer cells are selected from the group consisting of 4-isopropoxy-2-butanone, cyclohexanone, Dimethyl succinate, 1,3-bis(1,1-dimethylethyl)-benzene, diethyl ether, 2-methyl-2-hepten-6-one, 3-methyl-3-buten-1-ol, 4-methylbenzyl alcohol, and combinations thereof. In further embodiments, the set of VOCs, which are indicative of cell-to-cell signaling in cancer cells comprises 4-isopropoxy-2-butanone, cyclohexanone, dimethyl succinate, 1,3-bis(1,1-dimethylethyl)-benzene, diethyl ether, and 2-methyl-2-hepten-6-one. In additional embodiments, the set of VOCs, which are indicative of cell-to-cell signaling in cancer cells comprises 3-methyl-3-buten-1-ol and 4-methylbenzyl alcohol. In still further embodiments, the set of VOCs, which are indicative of cell-to-cell signaling in cancer cells comprises 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol and 4-methylbenzyl alcohol.

The term “cancer” refers to a disorder in which a population of cells has become, in varying degrees, unresponsive to the control mechanisms that normally govern proliferation and differentiation. Cancer refers to various types of malignant neoplasms and tumors, including primary tumors, and tumor metastasis. Non-limiting examples of cancers in which cell-to-cell signaling can be detected by the method of the present invention include brain, ovarian, colon, prostate, kidney, bladder, breast, lung, oral, and skin cancers. Specific examples of cancers are: carcinomas, sarcomas, myelomas, leukemias, lymphomas and mixed type tumors. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leiomyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

According to some currently preferred embodiments, the methods of the present invention are directed to detecting cell-to-cell signaling in lung cancer cells and/or diagnosing, prognosing and monitoring lung cancer. According to the principles of the present invention the term “lung cancer” relates to small cell lung cancers (SCLC) and non-small cell lung cancers (NSCLC) including, but not limited to, adenocarcinomas, adenosquamous carcinoma, squamous cell carcinomas, large cell carcinomas, bronchial carcinoids, cancers of supporting lung tissue, and mixtures of different types of NSCLC, at different stages, i.e., Stages I, II, III or IV. Each possibility represents a separate embodiment of the invention. Encompassed by this term are pre-cancerous conditions and metastasis to different sites. In certain embodiments, the lung cancer is selected from a non-small cell lung carcinoma and adenocarcinoma.

In some related embodiments, the at least one VOC, which is indicative of cell-to-cell signaling in cancer cells is indicative of cell-to-cell signaling in lung cancer cells. It has been surprisingly found by the inventors that at least some of the VOCs which are indicative of cellular communication in lung cancer cells are different from the previously known lung cancer VOC biomarkers. Accordingly, in certain embodiments, the set of VOCs as biomarkers of cell-to-cell signaling in cancer cells (also termed herein “signature set”), wherein said cancer cells are lung cancer cells, does not include at least one of the following VOCs: 3-methyl-butanal, 2-methyl-butanal, dodecane, and 3,8-dimethyl-undecane. In further embodiments, the set of VOCs as biomarkers of cell-to-cell signaling in cancer cells, wherein said cancer cells are lung cancer cells, does not include any one of the following VOCs: 3-methyl-butanal, 2-methyl-butanal, dodecane, and 3,8-dimethyl-undecane.

The methods of the present invention are ex-vivo methods which comprise the use of any sample or specimen. In some embodiments, the VOCs are measured directly through exposure to the sample. In other embodiments, the VOCs are measured through exposure to the headspace of a container in which the sample/specimen was placed. According to some embodiments, the test sample is selected from a headspace sample, exhaled breath, skin volatiles sample, and bodily fluid or secretion of the subject.

In certain embodiments, the test sample is a headspace sample. The headspace sample can be obtained from a container in which normal, cancerous or precancerous isolated cells and/or tissues of the subject have been deposited. In specific embodiments, the methods of the present invention are applicable for detecting at least one VOC from a container comprising a tissue sample. In some embodiments, the tissue sample comprises a histological section. In particular embodiments, the tissue sample comprises isolated cells. The number of isolated cells can range from a single cell to 10, 100, 1,000, 10,000, 100,000 or 1,000,000 cells. According to some embodiments, the container from which the headspace sample is obtained comprises about 1-10,000 cells. In further embodiments, the container comprises about 1-10 cells. In certain embodiments, the container comprises 1 cell. In additional embodiments, the container comprises 2 or 4 cells. According to some exemplary embodiments, the isolated cells comprise lung epithelial cells.

The headspace sample can further be obtained from a container wherein breath samples, bodily fluids or secretions have been deposited. Bodily fluids or secretions within the scope of the present invention include, but are not limited to, serum, urine, feces, sweat, vaginal discharge, saliva and sperm. Each possibility represents a separate embodiment of the present invention. Alternatively, the sample of bodily fluid or secretion can be analyzed directly to detect the at least one VOC indicative of cell-to-cell signaling.

The VOCS can be extracted from the headspace sample, as known in the art. One non-limiting example of a suitable extraction method includes In-Tube Extraction (ITEX).

In certain embodiments, the test sample comprises exhaled breath sample. The exhaled breath sample can be analyzed directly by suitable systems or devices, as described hereinbelow, by exposing said system or device to breath directly exhaled by the subject through a mouthpiece. In certain embodiments, the exposure is done directly, without a need for pre-concentrating or dehumidifying the sample.

Alternatively, the exhaled breath sample can be collected in any manner known to a person of ordinary skill in the art. In certain embodiments, the breath sample is collected using a breath collector apparatus. Specifically, the breath collector apparatus is designed to collect alveolar breath samples. Non-limiting examples of suitable breath collector apparatuses include apparatuses approved by the American Thoracic Society/European Respiratory Society (ATS/ERS); Silkoff et al., Am. J. Respir. Crit. Care Med., 2005, 171, 912.

In certain embodiments, the sample is pre-concentrated prior to the measurement of VOCs. Breath concentrators that are within the scope of the present invention include, but are not limited to,
Solid Phase Microextraction (SPME)—The SPME technique is based on a fiber coated with a liquid (polymer), a solid (sorbent), or combination thereof. The fiber coating extracts the compounds from the sample either by absorption (where the coating is liquid) or by adsorption (where the coating is solid). Non-limiting examples of coating polymers include polydimethylsiloxane, polydimethylsiloxane-divinylbenzene and polydimethylsiloxane-carboxen.
Sorbent Tubes—Sorbent tubes are typically made of glass and contain various types of solid adsorbent material (sorbents). Commonly used sorbents include activated charcoal, silica gel, and organic porous polymers such as Tenax and Amberlite XAD resins. Sorbent tubes are attached to air sampling pumps for sample collection. A pump with a calibrated flow rate in ml/min draws a predetermined volume of air through the sorbent tube. Compounds are trapped onto the sorbent material throughout the sampling period. This technique was developed by the US National Institute for Occupational Safety and Health (NIOSH).
Cryogenic Concentrations—Cryogenic condensation is a process that allows recovery of volatile organic compounds (VOCs) for reuse. The condensation process requires very low temperatures so that VOCs can be condensed. Traditionally, chlorofluorocarbon (CFC) refrigerants have been used to condense the VOCs. Currently, liquid nitrogen is used in the cryogenic (less than −160° C.) condensation process.

In certain embodiments, the test sample comprises a skin volatiles sample. The skin volatiles sample can be obtained and analyzed by placing a suitable sensor device directly on subject's skin. Alternatively, the skin volatiles sample can be collected by placing an absorbent material on the skin of the subject for a predetermined period of time. The VOCs can be further retrieved from the absorbent material, for example, by controlled heating.

The methods of the present invention involve collection of the test sample from a test subject. The term “test subject” as used herein refer a mammal, preferably a human. The diagnosis, prognosis and/or monitoring of cancer comprises the diagnosis of a subject who is at risk of developing cancer, a subject who is suspected of having cancer, or a subject who was diagnosed with cancer using commonly available diagnostic tests (e.g. biopsy or computed tomography (CT) scan). The present invention further provides the monitoring of cancer in patients having cancer. The term “monitoring” as used herein refers to the monitoring of disease progression or disease regression following treatment. Also encompassed by this term is the evaluation of treatment efficacy using the methods of the present invention.

In order to detect cell-to-cell signaling in cancer cells, the level of the at least one VOC from the test sample has to be compared to a reference value. As used herein, the terms “reference” and “reference value” are interchangeable and refer to a threshold criterion/value to which the measured VOCs levels are compared in order to detect cellular communication in cancer cells and/or to diagnose, prognose or monitor cancer in a subject. The reference value can be derived in any one of a number of manners. For example, the reference value may be based on a collection of data of samples comprising a headspace of at least two cell cultures or populations, which are co-cultured without a physical contact therebetween (also termed herein “co-culture headspace”). The cell cultures are, therefore, only gaseously connected through a mutual headspace (i.e., the same gas environment above the cells). The VOCs emitted from the mutual headspace of the two or more cell cultures can be monitored to identify and determine the levels of each VOCs present in the co-culture headspace. Preferably, the composition and relative abundance of the VOCs in said profile is recorded as a function of cells' co-culturing time and/or number of cells present in the at least two cell cultures. The co-culture headspace provides a medium in which the cells can interact without having a direct physical contact, wherein said interaction (i.e., cell-to-cell signaling) is reflected through a specific VOCs profile, including absence or presence of particular VOCs and specific levels of the VOCs, which are present in the co-culture headspace, as compared to a control. The obtained VOCs profiles can be compared with the VOCs emitted from the headspace of a mono-culture of each one of the at least two cell populations. The obtained VOCs profiles can be further compared with the VOCs profiles of the same at least two cell cultures, which were co-cultured by having a direct physical contact therebetween (also termed herein “direct contact co-culture”), e.g., incubated within a mutual growth medium. While the co-culture headspace includes VOCs, which are directly emitted from the cells, as well as VOCs, which originate from and are indicative of cell-to-cell signaling, the direct contact co-culture and mono-cultures include only VOCs, which are directly emitted from the cells. In this way, comparing the VOCs profile of the co-culture headspace with the direct contact co-culture and/or mono-culture allows to eliminate the VOCs, which are not associated with the cell-to-cell signaling in the co-culture headspace and to establish a signature set of VOCs, which are indicative solely of cellular communication.

In order to evaluate cellular communication of cancer cells, at least one of the at least two cell populations must include cancer cells. Cellular communication can occur between cancer cells and healthy cells, as well as among cancer cells, wherein the cancer cell types can be same or different.

According to some embodiments, the co-culture headspace comprises a first cell culture and a second cell culture. According to further embodiments, the first cell culture comprises cancer cells and the second cell culture comprises normal cells. According to some embodiments, the first cell culture comprises cancer cells and the second cell culture comprises cancer cells, which are identical to the cancer cells of the first cell culture. According to other embodiments, the first cell culture comprises cancer cells and the second cell culture comprises cancer cells, which are distinct from the cancer cell of the first cell culture. The term “distinct”, as used herein refers to different genetic mutations of the same cell type or to different cell types.

According to some embodiments, the first cell culture comprises cancer cells. In further embodiments, said cancer is a lung cancer. In yet further embodiments, said cancer comprises a p53 mutation. In certain embodiments, the p53 mutation is a A549 p53 mutation. In certain embodiments, the p53 mutation is a H1299 p53 mutation. In certain embodiments, the p53 mutation is a H1975 p53 mutation.

According to some embodiments, the second cell culture comprises normal cells. In further embodiments, said normal cells comprise lung cells. In certain embodiments, said normal cells are BEAS-2B cells.

According to some embodiments, the second cell culture comprises cancer cells. In further embodiments, the cancer cells are lung cancer cells. In yet further embodiments, said cancer comprises a p53 mutation. In certain embodiments, the p53 mutation is a A549 p53 mutation. In certain embodiments, the p53 mutation is a H1299 p53 mutation. In certain embodiments, the p53 mutation is a H1975 p53 mutation.

The reference value can be obtained from co-culture headspace sample comprising from about 1 to about 1,000,000 cells, from about 1 to about 100,000 cells, from about 1 to about 10,000 cells, from about 1 to about 1,000 cells, from about 1 to about 100 cells, or from about 1 to about 10 cells. Each possibility represents a separate embodiment of the present invention. According to some embodiments, the co-culture headspace sample comprises at least about 1,000 cells. In certain embodiments, the co-culture headspace sample comprises 1 cell. In additional embodiments, the co-culture headspace sample comprises 2 or 4 cells. In further embodiments, the co-culture headspace sample comprises about 10,000 cells. The reference value can be selected based on the number of cells in the test sample (wherein said test sample is obtained from a headspace of a container comprising isolated cells). In particular, the number of cells in the test sample and in the co-culture headspace sample can be identical or nearly identical (e.g., having less than 10% difference).

In some embodiments, the samples comprise a headspace of lung cancer cells. The samples can be obtained from lung cancer cells having various mutations. The level of the at least one VOC can be compared to a plurality of reference values, wherein each reference value is based on a collection of data of samples comprising a headspace of a specific type of lung cancer cells (e.g., cancer cells having a specific mutation).

The collection of data of samples from which the reference value is derived can comprise at least 2 samples, at least 5 samples, at least 10 samples, at least 50 samples, at least 100 samples, or more.

The reference value can be determined from a collection of data of samples by using various learning and pattern recognition algorithms. Said algorithms include, but are not limited to, unsupervised principal component analysis (PCA), discriminant function analysis (DFA), artificial neural networks (ANN) and support vector machine analysis (SVM).

The step of comparing the level of the at least one VOC to a reference value can also involve the use of various algorithms and/or statistical analysis techniques, in particular, when the VOC level in compared to a plurality of reference values. The level or concentration of the at least one VOC can be compared to a plurality of reference values, derived from different types of cancer cells and therefore corresponding to VOCs which are indicative of cellular communication between said specific cancer cells. Said comparison can be made to obtain a closest match between the VOC level and one of the plurality of reference values. The term “closest match”, as used herein, relates in some embodiments, to a difference between the level or concentration of the at least one VOC and a reference value, which is smaller than the difference between said level or concentration of the at least one VOC and any other reference value.

Additionally, the methods of the present invention can involve a step of identifying a plurality of VOCs, which are indicative of cell-to cell signaling. The term “plurality”, as used herein, means more than one. Said plurality of VOCs can be characterized by a pattern, wherein the level of each VOC in said pattern should be compared to a suitable reference value (which relates to this specific VOC). The VOCs pattern can be compared to a plurality of reference values with a pattern recognition analyzer which utilizes various algorithms, including, but not limited to, principal component analysis, Fischer linear analysis, neural network algorithms, genetic algorithms, fuzzy logic pattern recognition, and the like.

Non-limiting examples of suitable algorithms for comparing the level(s) of the VOC(s) to reference value(s) include artificial neural network (ANN) algorithm, support vector machine (SVM), discriminant function analysis (DFA), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MIR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, canonical discriminant analysis (CDA) and combinations thereof. Each possibility represents a separate embodiment of the invention.

According to the principles of the present invention, comparing the level of the at least one VOC being indicative of cell-to-cell signaling, to a reference value, provides detection of cellular communication in cancer cells and/or presence or absence of cancer, as well at the extent of cancer propagation. According to some embodiments, an essentially similar level of the at least one VOC, which is indicative of cell-to-cell signaling in the test sample as compared to the reference value is indicative of cell-to-cell signaling in cancer cells. According to some embodiments, an essentially similar level of the at least one VOC, which is indicative of cell-to-cell signaling in the test sample as compared to the reference value is indicative of cancer or provides the prediction of the test subject's response to a treatment regimen. In certain such embodiments, the reference is termed “positive reference” and is obtained from a collection of samples comprising cancer cells, which exhibit cell-to-cell signaling. According to some embodiments, the level of the at least one VOC is considered being essentially similar to the reference value, if it differs from the reference value by less than about 10%. According to further embodiments, the level of the at least one VOC is considered being essentially similar to the reference value, if it differs from the reference value by less than about 5%. According to yet further embodiments, the level of the at least one VOC is considered being essentially similar to the reference value, if it differs from the reference value by less than about 1%.

According to certain embodiments, a significantly similar level of the at least one VOC, which is indicative of cell-to-cell signaling in the test sample as compared to the reference value is indicative of cell-to cell signaling in cancer cells, is indicative of cancer, or provides the prediction of the test subject's response to a treatment regimen. Statistically significant similarity between the level or concentration of the at least one VOC or of each VOC from the plurality of VOCs and a corresponding reference value can be assessed by any test known to the person skilled in the art, e.g., an Equivalence test. According to some embodiments, the at least one VOC which is indicative of cell-to-cell signaling in cancer cells comprises a VOC that is present in a test sample in a level which is not more than one standard deviation [SD] larger or smaller than the positive reference value. More preferably, the level of the at least one VOC being indicative of cell-to-cell signaling in a test sample is not more than 0.5 or 0.25 standard deviation [SD] larger or smaller than the positive reference value.

According to some embodiments, a significantly different level of the at least one VOC, which is indicative of cell-to-cell signaling in the test sample as compared to the reference value is indicative of cell-to cell signaling in cancer cells. According to some embodiments, a significantly different level of the at least one VOC, which is indicative of cell-to-cell signaling in the test sample as compared to the reference value is indicative of cancer or provides the prediction of the test subject's response to a treatment regimen. In certain such embodiments, the reference is termed “negative reference” and is obtained from a collection of samples comprising cancer cells, which do not exhibit cell-to-cell signaling, e.g., headspace of a mono-culture or a co-culture having a physical contact between the at least two cell populations. The term “significantly different” as used herein refers to a quantitative difference in the level or concentration of the at least one VOC or of each VOC from the plurality of VOCs and a corresponding reference value.

A statistically significant difference can be determined by any test known to the person skilled in the art. Common tests for statistical significance include, among others, t-test, ANOVA1 Kruskal-Wallis, Wilcoxon, Mann-Whitney and odds ratio. Individual samples (of unknown status) can be compared with the negative reference. An increase or decrease in the level as compared to the negative reference value or a change, difference or deviation from the negative reference value, can be considered to exist if the level differs from the reference value, by about 5% or more, by about 10% or more, by about 20% or more, or by about 50% or more compared to the reference value. Statistical significance may alternatively be calculated as P<0.05. Methods of determining statistical significance are known and are readily used by a person of skill in the art. In a further alternative, increased levels, decreased levels, deviation, and changes can be determined by recourse to assay reference limits or reference intervals. These can be calculated from intuitive assessment or non-parametric methods. Overall, these methods calculate the 0.025, and 0.975 fractiles as 0.025* (n+1) and 0.975* (n+1). Such methods are well known in the art. The presence of a VOC marker in the test sample, which negative reference value is zero, is also contemplated as an increased level, deviation or change. The absence of a VOC marker in the test sample, which negative reference value is other than zero, is also contemplated as a decreased level, deviation or change. According to some embodiments, the at least one VOC which is indicative of cell-to-cell signaling in cancer cells comprises a VOC that is present in a test sample in a level which is at least one standard deviation [SD] larger or smaller than the negative reference value. More preferably, the level of the at least one VOC being indicative of cell-to-cell signaling in a test sample is at least 2 [SD] or 3 [SD] larger or smaller than the negative reference value.

The determination of the level of at least one volatile organic compound is performed, according to some embodiments of the present invention, by the use of at least one technique including, but not limited to, Gas-Chromatography (GC), GC-lined Mass-Spectrometry (GC-MS), Proton Transfer Reaction Mass-Spectrometry (PTR-MS), Electronic nose device (F-nose), and Quartz Crystal Microbalance (QCM). Each possibility represents a separate embodiment of the invention.

Gas Chromatography (GC) linked to mass spectrometry (MS) is often used to determine the chemical identity and composition of breath VOCs (Miekisch et al., Clinica Chimica Acta, 2004, 347, 25-39). In this set-up, the GC utilizes a capillary column having characteristic dimensions (length, diameter, film thickness) as well as characteristic phase properties. The difference in the chemical properties of different molecules in a mixture allows the separation of the molecules as the sample travels through the column, wherein each molecule has a characteristic time (termed retention time) in which it passes through the column under set conditions. This allows the mass spectrometer to capture, ionize, accelerate, deflect, and detect the ionized molecules separately. The MS signal is obtained by ionization of the molecules or molecular fragments and measurement of their mass to charge ratio by comparing it to a reference collection.

Proton transfer reaction-mass spectrometry (PTR-MS) is reviewed in Lindinger et al., Int. J. Mass Spectrom. Ion Process, 1998, 173, 191-241 and Lindinger et al., Adv. Gas Phase Ion Chem., 2001, 4, 191-241. Briefly, PTR-MS measures VOCs which react with H₃O⁺ ions that are added from an ion source. VOCs with a proton affinity that is larger than that of water (166.5 kcal×mol⁻¹) undergo a proton-transfer reaction with the H₃O⁺ ions as follows: H₃O⁺+R→RH⁺+H₂O. At the end of the drift tube reactor, a fraction of the ions is sampled by a quadrupole mass spectrometer, which measures the H₃O⁺ and RH⁺ ions. The ion signal at a certain mass is linearly dependent on the concentration of the precursor VOC in the sample air. In PTR-MS only the mass of VOCs is determined, causing some ambiguity in the identity of the VOCs. Thus, this technique does not allow a separate detection of different VOCs having the same mass. Further overlap of ion masses is caused by a limited degree of ion fragmentation and ion clustering in the drift tube.

Quartz Crystal Microbalance (QCM) is a piezoelectric-based device which can measure very small mass changes, mostly down to few nanograms. Briefly, QCM works by sending an electrical signal through a gold-plated quartz crystal, which causes vibrations in the crystal at a specific resonant frequency measured by the QCM. The resulted frequency shift can be translated to a change in mass on the QCM surface, mostly via using the Sauerbrey equation:

$$Δ f = \frac{- 2 {f}_{0}^{2}}{A \sqrt{{ρ}_{q} {μ}_{q}}} Δ m$$

This equitation is used to correlate changes in the oscillation frequency of a piezoelectric crystal (Δf) with the mass deposited on it (Δm). Other parameters which affect the signals are the resonant frequency (f₀), the area between electrodes of the piezo-electric crystal (A), density (ρ_{q}) and shear modulus (μQ) of quartz.

Electronic nose devices perform odor detection through the use of an array of broadly cross-reactive sensors in conjunction with pattern recognition methods (Rock et al, Chem. Rev., 2008, 108, 705-725). The sensors can include any type of electrically conducting or semi-conducting material, such as, but not limited to, metal, preferably, gold or platinum nanoparticles capped with organic ligands, carbon, e.g., carbon nanotubes coated with a suitable organic coating, silicon, polymer and any combinations thereof. In contrast to the “lock-and-key” approach, each sensor in the electronic nose device is broadly responsive to a variety of odorants. In this architecture, each analyte produces a distinct fingerprint from the array of broadly cross-reactive sensors. This allows to considerably widen variety of compounds to which a given matrix is sensitive, to increase the degree of component identification and, in specific cases, to perform an analysis of individual components in complex multi-component (bio) chemical media. Pattern recognition algorithms can then be used to obtain information on the identity, properties and concentration of the vapor exposed to the electronic nose device.

According to some embodiments, the step of identifying and determining the level of the at least one VOC from the test sample comprises the use of Gas-Chromatography-Mass Spectrometry (GC-MS) combined with In-tube Extraction (ITEX). In-tube extraction is a solventless extraction technique in which a headspace syringe with a microtrap (e.g., a needle body) filled with an adsorbent material is used. The analytes are extracted from sample headspace by dynamic extraction. The needle body is surrounded by a separate heater, which is used for thermal desorption of analytes into the injection port of a GC-MS system. Non-limiting examples of suitable adsorbent materials include Tenax TA, activated carbon, and polydimethylsiloxane. In some exemplary embodiments, the adsorption material is Tenax TA.

The diagnosing methods according to various aspects and embodiments of the invention can further comprise a step of treating the test subject if cancer is diagnosed. In some embodiments, treating the test subject comprises surgery, radiation therapy, chemotherapy, surveillance, adjuvant (additional) therapy, targeted therapy, or a combination of these treatments. In certain embodiments, the cancer is lung cancer.

According to another aspect, there is provided a method of identifying a set of volatile organic compounds (VOCs) indicative of cell-to-cell signaling in cancer cells, comprising the steps of: (a) providing a first cell culture comprising cancer cells and a second cell culture, wherein there is no physical contact between the first cell culture and the second cell culture; (b) co-culturing the first cell culture and the second cell culture under a mutual headspace; (c) determining the levels of VOCs in the mutual headspace; (d) comparing the levels of the VOCs in the mutual headspace to the levels of VOCs in a control sample; and (e) identifying a set of VOCs in the mutual headspace having levels that are significantly different as compared to the control sample.

The term “no physical contact”, as used herein, refers in some embodiments to the lack of direct contact between at least 90% of cells of the first cell culture and at least 90% of cells of the second cell culture. In further embodiments, the term “no physical contact” refers to the lack of direct contact between at least 95% of cells of the first cell culture and at least 95% of cells of the second cell culture. In yet further embodiments, the term “no physical contact” refers to the lack of direct contact between at least 99% of cells of the first cell culture and at least 99% of cells of the second cell culture. In still further embodiments, the term “no physical contact”, refers to the lack of a mutual growth medium between the first cell culture and the second cell culture. It is to be understood, though, that the first cell culture and the second cell culture are to be gaseously connected through a mutual headspace in step (b).

The first cell culture and the second cell culture can be cultured as known in the art, for example in two separate petri dishes held within a sealed container, a glass vial or a culture flask, as long as the two cell cultures do not physically contact each other within the vial or the flask. A physical barrier can be disposed between said two cell cultures. Preferably, the container in which the cells are co-cultured is adapted for automatic headspace sampling.

The cell cultures can be cultured in a growth medium, preferably a defined or synthetic medium, such as, for example, RPMI 1640, and supplemented with at least one of a blood serum, synthetic serum, and antibiotics.

Step (a) can further include providing additional cell cultures, including, e.g., a third cell culture, a fourth cell culture, and so on. Step (b) can include co-culturing said additional cell cultures together with the first cell culture and the second cell culture.

According to some embodiments, step (b) comprising co-culturing the first cell culture and the second cell culture under a mutual headspace comprises incubating the cell cultures for at least about 1 hour. According to further embodiments, step (b) comprises incubating the cell cultures for at least about 5 hours. According to yet further embodiments, step (b) comprises incubating the cell cultures for at least about 12 hours. According to still further embodiments, step (b) comprises incubating the cell cultures for at least about 24 hours. According to yet further embodiments, step (b) comprises incubating the cell cultures for at least about 48 hours. According to still further embodiments, step (b) comprises incubating the cell cultures for at least about 72 hours.

According to some embodiments, step (b) comprising co-culturing the first cell culture and the second cell culture under a mutual headspace comprises incubating the cell cultures for about 1-72 hours. According to further embodiments, step (b) comprises incubating the cell cultures for about 24-72 hours.

According to various aspects and embodiments of the present invention, the first cell culture comprises cancer cells. As cellular communication can occur between cancer cells and healthy cells, as well as among cancer cells, the second cell culture can contain healthy cells or cancer cells. In some embodiments, said cancer is a lung cancer. In further embodiments, said cancer comprises a p53 mutation. In certain embodiments, the p53 mutation is a A549 p53 mutation. In certain embodiments, the p53 mutation is a H1299 p53 mutation. In certain embodiments, the p53 mutation is a H1975 p53 mutation.

According to some embodiments, the second cell culture comprises normal cells. In further embodiments, said normal cells comprise lung cells. In certain embodiments, said normal cells are BEAS-2B cells.

According to some embodiments, the second cell culture comprises cancer cells, which are identical to the cancer cells of the first cell culture. According to other embodiments, the second cell culture comprises cancer cells, which are distinct from the cancer cell of the first cell culture. In some embodiments, the cancer cells are lung cancer cells. In further embodiments, said cancer comprises a p53 mutation. In certain embodiments, the p53 mutation is a A549 p53 mutation. In certain embodiments, the p53 mutation is a H1299 p53 mutation. In certain embodiments, the p53 mutation is a H1975 p53 mutation.

Each one of the first cell culture and the second cell culture can contain from about 1 to about 1,000,000 cells, from about 1 to about 100,000 cells, from about 1 to about 10,000 cells, from about 1 to about 1,000 cells, from about 1 to about 100 cells, or from about 1 to about 10 cells. Each possibility represents a separate embodiment of the invention. According to some embodiments, the first cell culture, the second cell culture or both comprises at least about 1,000 cells.

In certain embodiments, the first cell culture comprises 1 cell. In additional embodiments, the first cell culture comprises 2 or 4 cells. In further embodiments, the first cell culture comprises about 10,000 cells. In certain embodiments, the second cell culture comprises 1 cell. In additional embodiments, the second cell culture comprises 2 or 4 cells. In further embodiments, the second cell culture comprises about 10,000 cells. The number of cells in the first cell culture and the second cell culture can be same or different.

In some embodiments, when the first cell culture, the second cell culture or both comprise 1 cell, the co-culturing step is performed in a designated cell, which is configured to assess the communication through VOCs in a single cell resolution. According to some embodiments, the device comprises two culturing wells connected through Tesla's check valve that allows the headspace above cultured single cell to flow in one direction. The culturing wells can be connected by microchannels to two outlets for media perfusion purposes. In further embodiments, the device comprises miniaturized Tenax tubes, such that the headspace can be adsorbed thereon. In some embodiments, the device further comprises an air micropump.

Step (c) comprising determining the levels of VOCs in the mutual headspace can be performed by at least one of Gas-Chromatography (GC), GC-lined Mass-Spectrometry (GC-MS), and Proton Transfer Reaction Mass-Spectrometry (PTR-MS). According to some embodiments, step (c) is performed by means of GC-MS combined with In-tube Extraction (ITEX).

According to certain embodiments, each VOC present in the mutual headspace is identified and its level is measured.

The levels of VOCs can be monitored throughout the entire co-culturing process of step (b). In other words, step (c) can be performed within different time periods following step (b). In some embodiments, step (c) is performed within less than about 1 minute from the formation of a mutual headspace in step (b). In certain such embodiments, the co-culturing continues for less than about 1 minute. In further embodiments, step (c) is performed after at least about 24 hours from the formation of a mutual headspace in step (b). In certain such embodiments, the co-culturing continues for at least about 24 hours. In still further embodiments, step (c) is performed after at least about 48 hours from the formation of a mutual headspace in step (b). In certain such embodiments, the co-culturing continues for at least about 48 hours. In yet further embodiments, step (c) is performed after at least about 72 hours from the formation of a mutual headspace in step (b). In certain such embodiments, the co-culturing continues for at least about 72 hours.

According to various aspects and embodiments of the present invention, the method of identifying a set of VOCs indicative of cell-to-cell signaling in cancer cells comprises step (d) comprising comparing the levels of the VOCs in the mutual headspace to the levels of VOCs in a control sample.

As explained hereinabove, the VOCs contents of the mutual headspace and the VOCs levels result not only from the direct emission from the cells, but also from the interaction between the cells of the two cell cultures, wherein, for examples some VOCs can be consumed as a result of cellular interaction. Comparing the VOCs of the mutual headspace with VOCs of each one of the cell cultures grown independently (i.e., not having a mutual headspace) would therefore allow to pick out the VOCs which result specifically from cellular communication, which have a physical barrier therebetween. The levels of the VOCs found in the mutual headspace of the first cell culture and the second cell culture having no physical contact therebetween can further be compared to VOCs emitted from a combination of said two cell cultures grown under a mutual headspace and having a physical contact therebetween.

The control sample can therefore be selected from (a) a headspace of the first cell culture (also termed herein “first cell mono-culture”), (b) a headspace of the second cell culture (also termed herein “second cell mono-culture”), (c) a headspace of the first cell culture and the second cell culture, wherein there is a physical contact between the first cell culture and the second cell culture (also termed herein “direct contact co-culture”), and any combination thereof. The type of the cells (i.e., type of cancer mutation) in the co-culture should be identical to the type of the cells of the control sample. The number of cells in the co-culture and the control sample can be matched. For example, if the first cell culture and the second cell culture each contain about 10,000 cells, the control sample can also contain about 10,000 respective cells.

According to some embodiments, the first cell mono-culture comprises cancer cells. In further embodiments, said cancer is a lung cancer. In yet further embodiments, said cancer comprises a p53 mutation. In certain embodiments, the p53 mutation is selected from a A549 p53 mutation, H1299 p53 mutation, and H1975 p53 mutation. Each possibility represents a separate embodiment of the present invention. According to some embodiments, the first cell mono-culture comprises normal cells. In further embodiments, said normal cells comprise lung cells. In certain embodiments, said normal cells are BEAS-2B cells.

According to some embodiments, the second cell mono-culture comprises cancer cells. In further embodiments, said cancer is a lung cancer. In yet further embodiments, said cancer comprises a p53 mutation. In certain embodiments, the p53 mutation is selected from a A549 p53 mutation, H1299 p53 mutation, and H1975 p53 mutation. Each possibility represents a separate embodiment of the present invention. According to some embodiments, the first cell mono-culture comprises normal cells. In further embodiments, said normal cells comprise lung cells. In certain embodiments, said normal cells are BEAS-2B cells.

According to some embodiments, the direct contact co-culture comprises cancer cells, normal cells or any combination thereof. In certain embodiments, the direct contact co-culture comprises a co-culture of two different types of cancer cells. In certain embodiments, the direct contact co-culture comprises a co-culture normal cells and cancer cells. In further embodiments, said cancer is a lung cancer. In yet further embodiments, said cancer comprises a p53 mutation. In certain embodiments, the p53 mutation is selected from a A549 p53 mutation, H1299 p53 mutation, and H1975 p53 mutation. Each possibility represents a separate embodiment of the present invention. In certain embodiments, the normal cells comprise lung cells. In certain embodiments, said normal cells are BEAS-2B cells.

The control sample can further include a headspace of a third cell mono-culture, fourth cell mono-culture, and so on, in accordance with the number of cell cultures in steps (a) and (b) of the present method. The direct contact co-culture can further comprise a third or a fourth cell culture in direct contact with the first and the second cell cultures.

In certain embodiments, the level of at least one volatile organic compound in the mutual headspace of the co-culture is significantly increased as compared to the level of said compound in a control sample. According to other embodiments, the level of at least one volatile organic compound in the mutual headspace of the co-culture is significantly decreased as compared to the level of said compound in a control sample. According to a specific embodiment, the at least one VOC is a hydrocarbon and its level in the mutual headspace of the co-culture is significantly decreased as compared to the level of said compound in a control sample comprising a respective cell mono-culture. According to an additional specific embodiment, the at least one VOC is a ketone and its level in the mutual headspace of the co-culture is significantly increased as compared to the level of said compound in a control sample comprising a respective cell mono-culture.

In particular embodiments, the mutual headspace comprises a plurality of VOCs, which levels form a pattern. In further embodiments, said pattern is significantly different from the pattern of said volatile organic compounds in the control sample.

As mentioned hereinabove, the set of VOCs indicative of cell-to-cell signaling in cancer cells is determined by the distributions of VOCs in the mutual headspace of the co-culture of the first cell culture and the second cell culture in comparison to the distributions of the same VOCs in a control sample. The set of VOCs comprises specific VOCs for which a statistically significant difference in their level in the mutual headspace as compared to control samples exists. A statistically significant difference can be determined as described in detail hereinabove. Non-limiting examples of suitable statistical significance tests include t-test, ANOVA1 Kruskal-Wallis, Wilcoxon, Mann-Whitney and odds ratio. An increase or decrease in the VOC level as compared to a control or mean control level (determined from multiple identical control samples) or a reference value obtained from the control sample(s), or a change, difference or deviation from a control or reference value, can be considered to exist if the level differs from the control level or reference value, by about 5% or more, by about 10% or more, by about 20% or more, or by about 50% or more compared to the control level or reference value. When using mono-cultures as a control sample, their VOCs levels need to be appropriately combined to obtain a reference value. In some embodiments, an average of the VOCs profiles of the two mono-cultures is generated.

Statistical significance may alternatively be calculated as P<0.05 or P<0.01. In a further alternative, increased levels, decreased levels, deviation, and changes can be determined by recourse to assay reference limits or reference intervals. These can be calculated from intuitive assessment or non-parametric methods. Overall, these methods calculate the 0.025, and 0.975 fractiles as 0.025* (n+1) and 0.975* (n+1). Such methods are well known in the art. The presence of a VOC marker which is absent in a control sample, is also contemplated as an increased level, deviation or change. The absence of a VOC marker which is present in a control, for example, is also contemplated as a decreased level, deviation or change.

The reference value used in the methods of detecting cell-to-cell signaling and/or diagnosing, monitoring or prognosing cancer in a subject is also termed herein “first reference value” and the reference value used in the method of identifying a set of volatile organic compounds (VOCs) indicative of cell-to-cell signaling in cancer cells is also termed herein “second reference value”.

According to some embodiments, the set of volatile organic compounds which is indicative of cell-to-cell signaling in cancer cells comprises VOCs that are present in the mutual headspace in levels which are at least one standard deviation [SD] larger or smaller than their mean level in a control sample comprising a first cell mono-culture combined with their mean levels in a control sample comprising a second cell mono-culture. More preferably, the levels of VOCs in the mutual headspace are at least 2 [SD] or 3 [SD] larger or smaller than their mean level in the mono-culture samples, as combined.

According to some embodiments, the set of volatile organic compounds which is indicative of cell-to-cell signaling in cancer cells comprises VOCs that are present in the mutual headspace in levels which are at least one standard deviation [SD] larger or smaller than their mean level in a control sample comprising a direct contact co-culture. More preferably, the levels of VOCs in the mutual headspace are at least 2 [SD] or 3 [SD] larger or smaller than their mean level in the direct contact co-culture.

According to the principles of the present invention, the VOCs, which levels in the mutual headspace are significantly different than in the control sample are indicative of cell-to-cell signaling.

Alternatively, the set of VOCs is characterized by a pattern which significantly differs from the patterns of said VOCs in control samples. The difference in the pattern can be analyzed with a pattern recognition analyzer which utilizes various algorithms including, but not limited to, principal component analysis, Fischer linear analysis, neural network algorithms, genetic algorithms, fuzzy logic pattern recognition, and the like. Non-limiting examples of suitable algorithms are artificial neural network (ANN) algorithm, support vector machine (SVM), discriminant function analysis (DFA), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PIS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, canonical discriminant analysis (CDA) and combinations thereof. Each possibility represents a separate embodiment of the invention.

According to some embodiments, the VOCs levels or the VOCs pattern are time-dependent. According to additional embodiments, the VOCs levels or the VOCs pattern depend on the number of cells in the first cell culture and the second cell culture. The signature sets of VOCs indicative of cell-to-cell signaling in cancer cells can therefore be classified not only according to the type of cancer cells but to the length of co-culturing (or incubation) process and the number of cells in the sample. A database containing such VOCs sets can be assembled to be used for the determination of cell-to-cell signaling in test samples and for diagnosing, prognosing or monitoring cancer in a test subject, wherein the VOCs set used for identification purposes can be chosen in accordance with the nature of the test sample and its handing process. Various VOCs sets can further be used during analysis of the sample by pattern recognition analyzer to select the closest match between the variety of VOCs sets and the VOCs of the test sample.

According to some currently preferred embodiments, the set of VOCs comprises at least one VOC selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, 4-methylbenzyl alcohol, 2-fluoro-acetamide, ethanol, fumaronitrile, 1,3-bis(1,1-dimethylethyl)-benzene, 1,3-benzodioxole-2-carboxylic acid, ethyl ester, 5-(dimethylamino)-1,3-benzenedicarboxylic acid, and combinations thereof. In some embodiments, the set of VOCS which are indicative of cell-to-cell signaling in cancer cells comprises at least two VOCs from the above list. In further embodiments, the set of VOCS which are indicative of cell-to-cell signaling in cancer cells comprises at least three VOCs from the above list, at least four VOCs, at least five VOCs, at least six VOCS, at least seven VOCs, at least eight VOCs, at least nine VOCs, or at least ten VOCs. Each possibility represents a separate embodiment of the invention.

According to certain embodiments, the VOCs, which are indicative of cell-to-cell signaling in cancer cells are selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, 4-methylbenzyl alcohol, and combinations thereof. In certain embodiments, the VOCs, which are indicative of cell-to-cell signaling in cancer cells are selected from the group consisting of 2-fluoro-acetamide, ethanol, fumaronitrile, 1,3-bis(1,1-dimethylethyl)-benzene, 1,3-benzodioxole-2-carboxylic acid, ethyl ester, 5-(dimethylamino)-1,3-benzenedicarboxylic acid, and combinations thereof. In certain embodiments, the VOCs, which are indicative of cell-to-cell signaling in cancer cells are selected from the group consisting of 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol, 4-methylbenzyl alcohol, and combinations thereof. In further embodiments, the set of VOCs, which are indicative of cell-to-cell signaling in cancer cells comprises 4-isopropoxy-2-butanone, cyclohexanone, dimethyl succinate, 1,3-bis(1,1-dimethylethyl)-benzene, diethyl ether, and 2-methyl-2-hepten-6-one. In additional embodiments, the set of VOCs, which are indicative of cell-to-cell signaling in cancer cells comprises 3-methyl-3-buten-1-ol and 4-methylbenzyl alcohol. In still further embodiments, the set of VOCs, which are indicative of cell-to-cell signaling in cancer cells comprises 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol and 4-methylbenzyl alcohol.

As used herein and in the appended claims the singular forms “a”, “an,” and “the” include plural references unless the content clearly dictates otherwise. Thus, for example, reference to “a sample” includes a plurality of such samples and so forth. It should be noted that the term “and” or the term “or” are generally employed in its sense including “and/or” unless the content clearly dictates otherwise.

As used herein, the term “about” refers to a range of values ±20%, or ±10%, or ±5% of a specified value.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Co-Culture System Setups

To evaluate growth kinetics and cell-to-cell signaling between cancer cells and cancer and healthy cells, cancer and normal cell lines genetically different in p53 status have been used, including A549, H1299, and H1975 human lung cancer lines and BEAS-2B normal cell lines. All the cell lines were cultured in RPMI medium and supplemented with 10% fetal bovine serum (FBS), and grown at 37° C. under 5% CO₂ and 95% relative humidity. The cells were allowed to reach 80-90% confluence prior to seeding. The culture medium was replenished with fresh medium every 2 or 3 days. Upon reaching 80-90% confluency, the cells were washed with phosphate buffer solution (PBS) and detached with 0.25% EDTA (Sigma-Aldrich) and then the cell suspensions were collected and centrifuged at 1000 rpm for 5 min. Following aspiration of the supernatant, the cell pellets were re-suspended and each cell type suspension was diluted at a density of 1×10⁴ cells/mL in the culture medium. The prepared cells (1×10⁴ cells) were seeded on the glass petri dish (35 mm) as described below and incubated at 37° C., 5% CO₂ for a period of 24, 48, and 72 hours (T₂₄, T₄₈ and T₇₂ h, respectively).

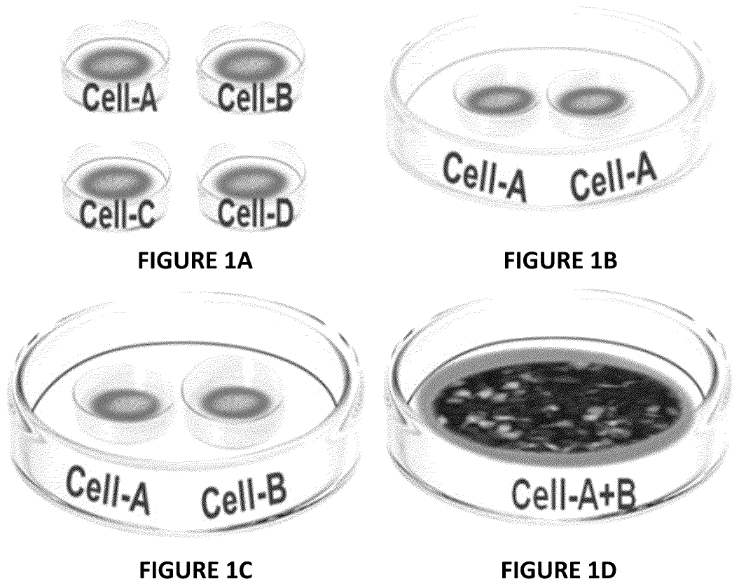

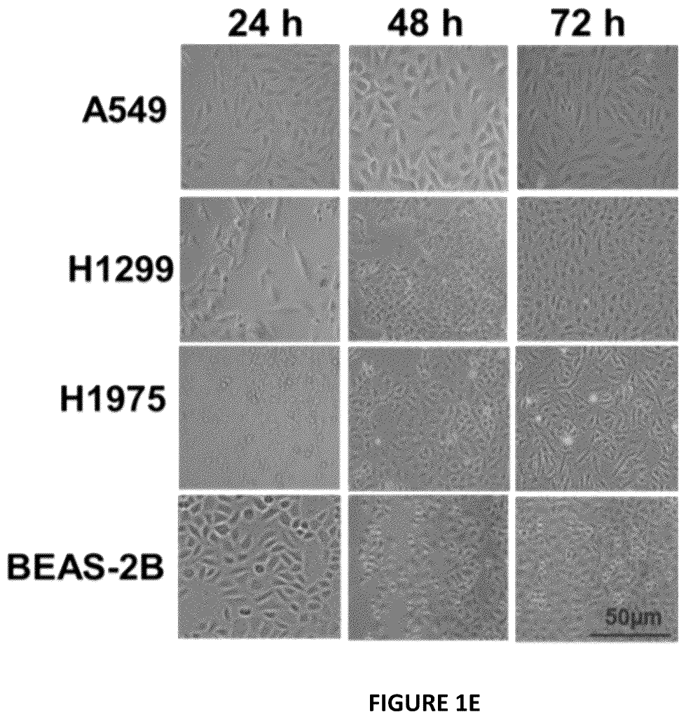

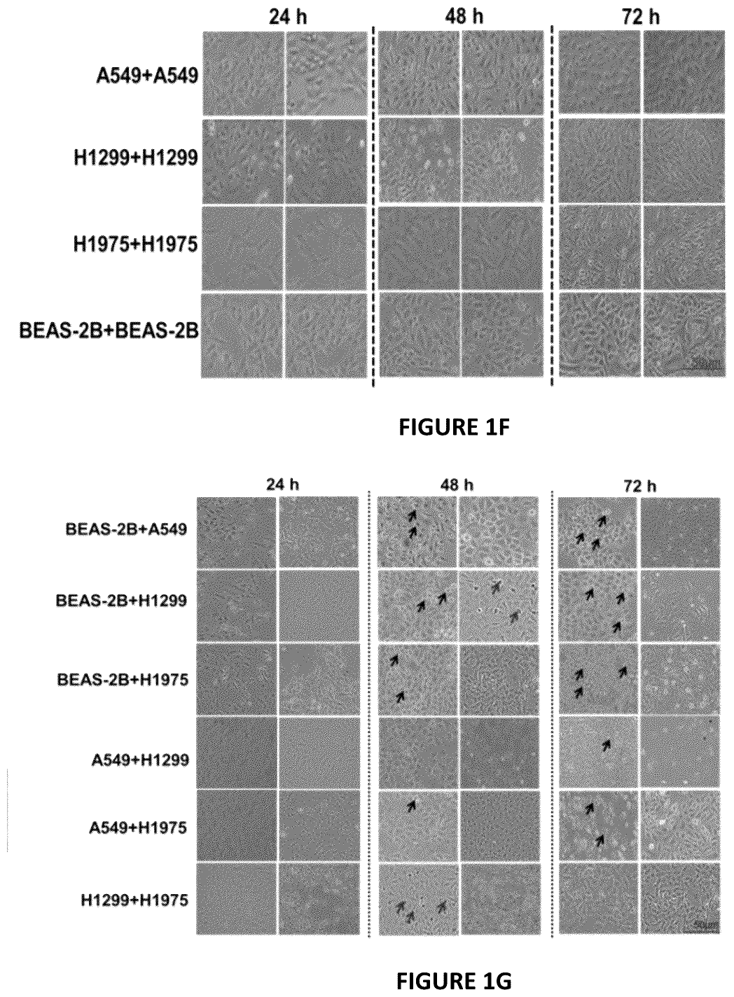

Petri dish co-culture experiments were performed in four different formats, as graphically described in FIGS. 1A-1D:
1. Format-1: Mono-culture format, wherein each of the cell cultures was seeded in a separate petri dish and cultured under its own headspace, without any physical, fluid or gaseous contact therebetween (FIG. 1A), including: Group-1: (A549), Group-2: (H1299), Group-3: (H1975), Group-4: (BEAS-2B).
2. Format-2: Same-cell pair co-culture headspace (co-culture headspace), wherein same type of cells were seeded in different petri-dishes and co-cultured under a mutual headspace (FIG. 1B), including: Group-5: (BEAS-2B+BEAS-2B), Group-6: (A549+A549), Group-7: (H1299+H1299), Group-8: (H1975+H1975).
3. Format-3: Different-cell pair co-culture headspace (co-culture headspace), wherein different cell types were seeded in two separate glass petri-dishes and co-cultured under a mutual headspace (FIG. 1C), including: Group-9: (BEAS-2B+A549), Group-10: (BEAS-2B+H1299), Group-11: (BEAS-2B+H1975), Group-12: (A549+H1299), Group-13: (A549+H1975), Group-14: (H1299+H1975).
4. Format-4: Different-cell pair co-culture having physical contact (direct contact co-culture), wherein different cell types were seeded together in one glass petri-dish and co-cultured under a mutual headspace (FIG. 1D), including: Group-15: (A549 & BEAS-2B), Group-16: (A549 & H1299), Group-17: (A549 & H1975), Group-18: (BEAS-2B & H1299), Group-19: (BEAS-2B & H1975), Group-20: (H1299 & H1975).

### Example 2: Morphological Changes in Co-Culture System Setups

Morphological changes of the cells in the first three of the experimental formats described in Example 1 were determined under a microscope (Korpi. A, Appl. Environ. Microbiol. 1998, 64, 2914). The morphological appearance of co-culture cells was compared with the mono-culture. Each experiment was done in duplicates at each of the three time points (T₂₄, T₄₈ and T₇₂). After incubation, the cell morphology of the mono-culture and same-cell pair co-culture headspace groups has shown insignificant changes (FIGS. 1E-1F) at all time points. In contrast, significant morphological changes (e.g., circular and floating cells) were observed in co-cultured combinations of different cell types in a time dependent manner. FIG. 1G shows a wide variety of morphology changes observed in the different-cell pair co-culture headspace experimental format. For example, when co-culturing normal cells with cancer cells under the same headspace, normal cells became circular and round shape, compared with control (mono-culture) after T₄₈ and T₇ h (FIG. 1G). While the H1975M cells in co-cultured pair of H1299⁻+H1975^{M} and A549^{W}+H1975^{M} combinations, showed insignificant morphological changes, H1299⁻ and A549^{W} cells exhibited changes in cell morphology into round shapes and cell shrinkage, compared with control (mono-culture) at T₇₂ (FIG. 1G). Co-culture pair combinations of cancer cells showed strong morphological changes on H1299 and normal cell lines.

The cells in the abovementioned experimental setups were further examined by digital imagery with traditional flow cytometry (ImageStream) (Megjugorac, N. J. et al., Immunol Invest. 36, 739 (2007)). For this analysis, Cell-A culture was labeled with DiO and Cell-B was labeled with DiD and incubated for 20 min. Then, cells were washed and co-cultured at a 1:1 ratio for T48 h. Immediately after incubation, cells were sent to ImageStream for analysis. On the ImageStream, each cell was simultaneously imaged in Hoechst (435-505 nm), DiI (505-560 nm), DiO (435-505 nm), DiD (642-745 nm) and bright-field (660-720 nm). Quantitative measurement of apoptotic nuclear morphology (e.g., increased nuclear texture and fragmentation) was performed using IDEAS software. The area of the bright regions of the nuclear image and the intensity of small bright nuclear details within IDEAS were found to be characteristic for apoptotic nuclei. The analysis has shown that physically unconnected co-culture of different cells as well as physically connected co-culture of different cells presented apoptotic and nuclear changes compared to mono-culture and headspace of physically unconnected co-culture cells of the same type.

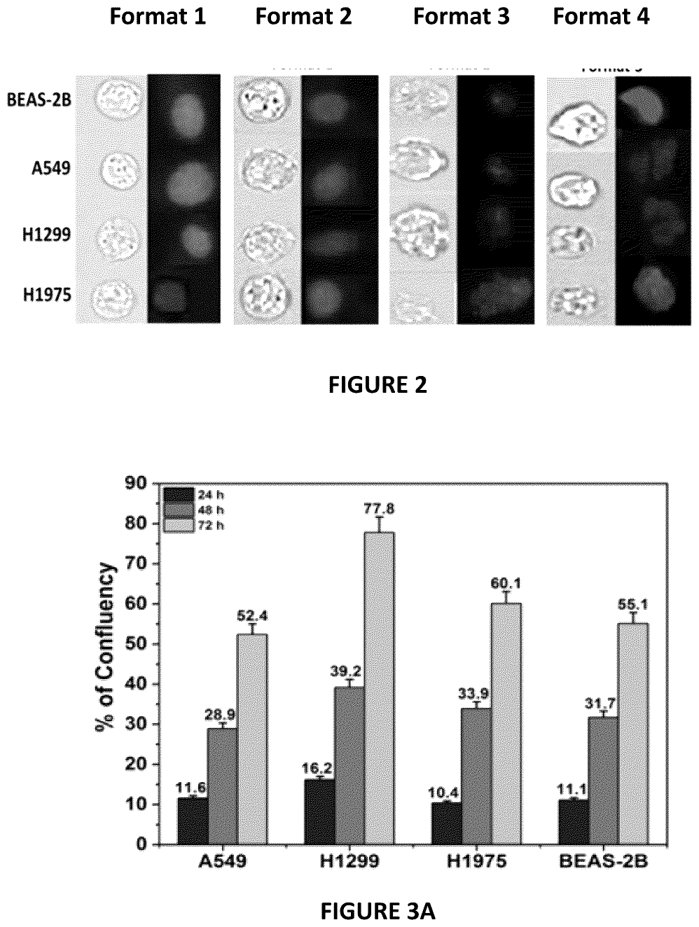

The ImageStream analysis of different type co-cultured cells at T₄₈ h showed reduced nuclei number of Hoechst stained cells (FIG. 2). Direct contact cell groups showed different size and shape of nuclei compared to control (mono-culture) groups. Intensity analysis with software-based nucleus shape and nucleus staining indicated significant changes in nuclear morphology. The Hoechst dye stained morphologically normal nuclei exhibited dimly blue, whereas co-culture physically unconnected cells (Format-2) and co-culture physically connected cells (Format-3) presented changes in nuclear morphology (nuclear shrinkage) exhibited bright blue and smaller nuclei after T₄₈ h (FIG. 2). These results show that two different cell types in co-culture induce morphological changes and apoptotic cell death due to soluble factors like chemical signals. But most interestingly it further suggests that such induction could also happen via volatile signals (headspace co-culture of unconnected cells). The rate of apoptosis was further verified by Hoechst staining, which detects chromatin condensation, one of the hallmarks of apoptotic cell death (Foghi, A. et al., Endocrinology, 139, 2041 (1998)).

ImageStream analysis of apoptotic cell death in different co-culture formats at T₀ and T₄₈ h was carried out (Table 1). Low apoptotic rate was observed when cells were cultured alone (1.8%). This rate was marginally increased (3.5%) in same-cell pair combinations at T₄₈ h. BEAS-2B cells co-cultured with other cell types showed 3% increase in apoptosis rate of unconnected cells, while in physically connected cell groups apoptosis levels were significantly (p<0.05) increased (47.1%) at T₄₈ h (Table 1). The high apoptotic rate in the physically connected co-cultured cells may be due to cell type but most likely not related to the high cell density, because at mono-culture cells (also with high cell density) apoptotic rate was lower (1%). Co-culturing H1299 cells directly with H1975 at T₄₈ h exhibited 54.1% cancer cell apoptosis, at the time co-culturing H1299 cells with A549 cells exhibited 21.2% rate at T₄₈ h (Table 1). When BEAS-2B and H1299 cells were co-cultured in an environment that prevents physical contact between the co-cultures, the apoptosis was drastically increased and it was almost 3 to 4-fold higher at T₄₈ h compared to T₀ h and to the same pair unconnected co-culture. Interestingly, H1975 cancer cell line showed the lowest apoptosis rate (2.2%) upon direct contact co-culture with H1299 cells at T₄₈ h. Apoptosis of H1299 and BEAS-2B cells was induced to a larger extent by direct contact co-culturing with H1975 and A549 cells as compared to the co-culture headspace setup, wherein the cells are not physically connected (Table 1). Analysis of the imaging flow cytometry results showed moderate changes in unconnected same-pair co-cultures. Direct contact co-culture of BEAS-2B and H1299 cells with H1975 cells showed high levels of morphological changes and apoptosis (54.1% and 51.5%), respectively. Without wishing to being bound by theory or mechanism of action, these results suggest that the proliferative and apoptotic effects of H1975 and A549 cells, is probably mainly induced by soluble factors which are dependent on direct cell contact between the cells cultured together, but most interestingly, it is also affected by volatile signals, although to a lower extent.

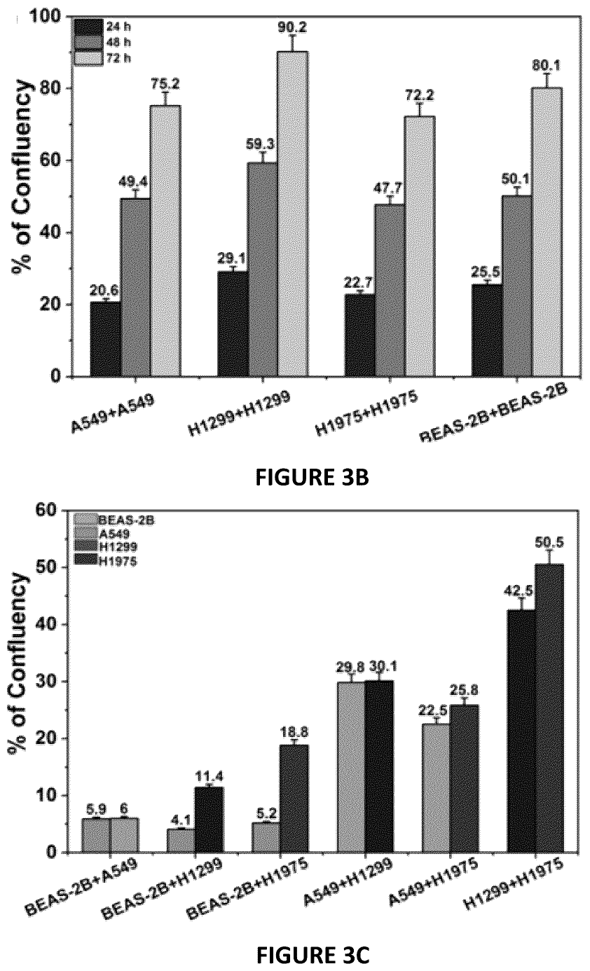

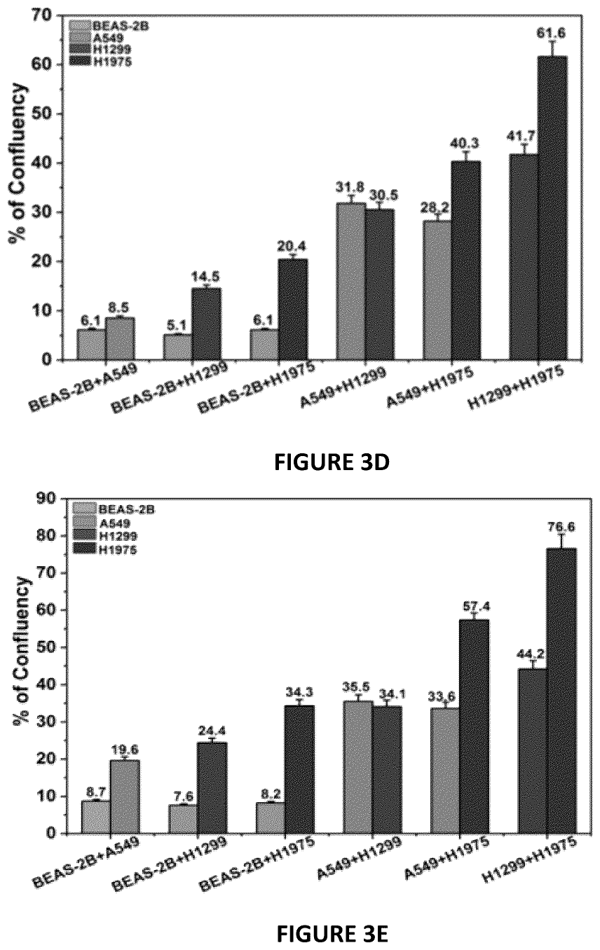

In parallel, cell growth after T₇₂ h was examined. Cell confluency in all combinations was measured by tryphan blue. Cell confluency was significantly (p<0.05) increased in a time dependent manner within each type of same-cell pair combinations of Format-2 (A549+A549 (75.2%), H1299+H1299 (90.2%) and BEAS-2B+BEAS-2B (80.1%)), as well as in comparison with the Format-1 setup, including mono-culture of the corresponding cell lines (FIGS. 3A-3B). In contrast, co-culture of physically unconnected different-cell pairs (Format-3) showed lower cell confluency than in same-cell type co-culture combinations at all time points (FIGS. 3C-3E). However, cell confluency rates of unconnected different-cell pairs were higher at T₇₂ h (FIG. 3E) than the mono-culture groups (FIG. 3A).

Without wishing to being bound by theory or mechanism of action, it is contemplated that since the kinetic and morphological characteristics of cells exhibited significant changes in the co-cultures, which are not physically connected, but their only possible way of contacting is through a mutual headspace (Formats 2 and 3), it is reasonable to assume that the main cause for the observed morphological and growth rate changes is the cross-talk, which occurs via the gas phase in the headspace above the cells.

### Example 3: VOCs Analysis of Co-Culture System Setups-Methodology

To explore the potential role of VOCs in the cell-to-cell cross-talk, the same experimental formats described in Example 1 were used.

### Sample Preparation

A549, H1299, H1975 and BEAS-2B cell lines were maintained in RPMI 1640 medium. In addition, 10% fetal bovine serum and 1% penicillin and streptomycin were added to the RPMI. The cells were grown to 40-60% confluent monolayer in the 75 cm² culture flask under standard conditions at 37° C. and 5% CO₂. After 15-24 h, the medium was removed and washed twice with pre-warmed medium without FBS and FBS-free medium was added to the flask and incubated for 15-20 h for starvation, then the cells were harvested using 0.25% trypsin-EDTA and the cell suspension was transferred into centrifuge tubes to prepare a series of 10 fold dilutions. Then, 1 μL of proper dilution was seeded (2 and 4 cells in different vials) in 2 ml, glass vial (flat bottom headspace vial) and the presence of a single cell was confirmed by microscopy. Thereafter, 500 μL 0.5% FBS growth medium was added to each vial and the samples were transferred for incubation. Seven biological replicates of each cell type were prepared for GC-MS analysis. Prior to the GC-MS analysis, the vials were sealed with magnetic crimp caps for 2 h (37° C. and 5% CO₂) to boost the accumulation of species released by the cells and to block the gas exchange with the ambient air. For VOCs measurement, all cell lines and control medium were incubated for T₀, T₂₄ and T₄₈ h. After incubation, sample and control medium (without cells) vials were immediately transferred for GC-MS analysis. Bulk cell samples were prepared similarly with some adjustments. 1×10⁴ cells were seeded in 2 ml, glass vial as described in FIGS. 1A-1D instead of petri dishes and incubated for 24, 48 and 72 h. After 48 h incubation period, no dead cells were observed in any of the cell lines. After 72 h, some floating cells were observed and the culture medium had turned red (all cell lines). Thus, the culture conditions of the 24, 48 and 72 h incubation periods ensured that the release of VOCs into the medium was mostly due to living cells.

### GC-MS Analysis

GC-MS analysis was performed using Agilent 7890B series GC system (Agilent, USA) connected to an Agilent 5977A mass selective detector (MSD) (Agilent, USA) and a PAL auto-sampler (Auto-PAI-RSI 120) equipped with an extractor EI source. The analytical column was a SLB-5 ms capillary column (with 5% phenyl methyl siloxane; 30 m in length; 0.25 mm in internal diameter; 1 μm in thickness; from Sigma-Aldrich). Ultra-high purity (99.999%) helium was used as carrier gas (flow-rate 1 mL/min). The GC was operated under the following temperature program: initially at 35° C., held for 10 min at 200° C., held at 240° C. for 21 min, ramped at 15° C. min-1 to 260° C., and held at 260° C. for 2 min, giving a total run time of 25.7 min.

### In-Tube Extraction (ITEX) Method

In-tube extraction device (ITEX) combined with the GC-MS system was used for headspace sampling. The sample vial was set on an automatic sampling system connected to the GC-MS (Auto-PAL-RSI 120). Automated ITEX applied a 1.3 ml, headspace syringe with a Tenax TA-filled needle body. The analytes were extracted from sample headspace by dynamic extraction onto the absorbent. The needle body was surrounded by a heating unit, which is used for analyte desorption into the injection port of a GC-MS. The auto-sampler was equipped with a single magnet mixer (SMM) and a temperature-controlled tray holder. The samples were placed in the tray cooler at 25° C.; after transfer to the SMM, the sample was heated (80° C.) and stirred at 500 rpm for 20 min to reach the extraction temperature of 80° C. to establish equilibrium distribution of the analytes between liquid and gas phase in the vial before extraction. The extraction volume of the gas phase was set to 1000 μL and 750 extraction strokes were used for the optimized method for each sample. The extraction flow-rate during extraction was set at 100 μL/sec. After the extraction, the sample vial was moved back to the tray. Desorption was performed by heating the ITEX trap to 250° C. with desorption flow rate of 1 mL purge gas, which was used to desorb and purge the extracted VOC of the sample at a flow-rate of 10 μL/sec into the hot injector. After desorption, the ITEX device was flushed with nitrogen gas at 260° C. for 5 min. Afterwards, the plunger was moved down, and the temperature was set to 80° C., to prepare the trap for the next extraction. The whole process (including injection, trap cleaning, and extraction of the following sample) was completed within the runtime of the GC oven program with cooling for about 5 h. An internal standard mixture (EPA-542) 1,4-Dichloro benzene-D4 was added (7 ppb) along with test samples as well as control medium to ensure that the GC-MS was functioning effectively. The test was based on examination of the retention time and peak shape of the solvents used in the calibration mixture.

### GC-MS Data Processing

The GC-MS chromatograms were analyzed using Mass Hunter qualitative (version B.07.00; Agilent Technologies, USA) analysis. The compounds were tentatively identified through spectral library match NISTL.14 (National Institute of Standards and Technology, USA). Qualitative analysis involved the area under the curve values; subtracting relevant media only headspace controls values (collected during the same experiment conditions). All experiments were repeated 7 times and the results expressed as the mean±standard deviation.

### Calibration

For the quantification of compounds detected in cells and medium solutions, external standard calibration was performed. For each VOC, pure standards were purchased (Sigma-Aldrich, MI, USA). The reagents stock solutions were made to a concentration of 1M by dissolving them in 1 ml, methanol. Calibration solutions of 1, 10, 50, 100 and 150 ppb were prepared. Standard curves were created based on the peak areas, which were obtained from Mass Hunter Qualitative analysis. The data were analyzed in triplicate.

### Data Collection and Analysis.

To identify significant differences in VOCs between the groups, t-test, Kruskal-Wallis test and an extension of the Wilcoxon rank-sum test to more than 2 groups was used. The patterns of the significant VOCs were confirmed using SAS JMP, Verison.12.0 (SAS Institute, Cary, North Carolina, USA; 1989, 2005). Data are presented as mean±SD, and a p<0.05 was considered statistically significant.

### Example 4: VOCs Analysis of Co-Culture System Setups Including Bulk Cell Samples-Results

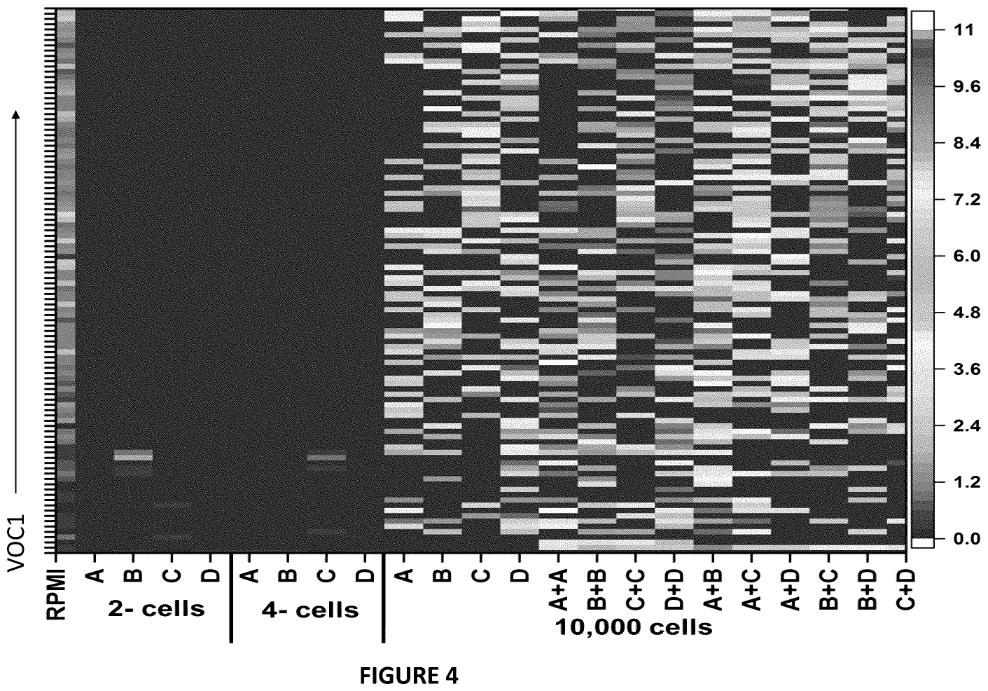

A total of 104 VOCs were significantly identified all together in the four experimental formats, which included bulk cell samples (1×10⁴ cells). Result show that mono-cultured groups exhibit different VOCs profile composition from that of the co-culture pair combination groups (FIG. 4 and Tables 2-4). A higher number of VOCs were detected in the co-culture pair combinations of the tested cell lines (FIG. 4). It can be clearly seen that in the same cell unconnected co-culture there was a higher abundance of VOCs. Without wishing to being bound by theory or mechanism of action, it is contemplated that the higher number of detected VOCs is due to the combination of the VOCs mixture of these four cell line isolates.

In Format-1 setup, which includes four different mono-cultures, after T₂₄ h incubation period, there was a difference in the peak area of 23 compounds in all cells as compared with the control, whereby concentrations of 20 VOCs were found to be significantly increased and of three compounds (2,5-Dimethyl-1-heptene; 2,5-Dimethyl-2,5-hexanediol; 4,6-Dimethyl-2-heptanone) were found to be significantly decreased (p<0.05) (Table 2). After T₄₈ h incubation period, differences in the peak area of 23 VOCs between all cell lines and control medium were detected, whereby concentrations of 22 VOCs were significantly increased and concentration of one VOC was significantly decreased (p<0.05). At T₇₂ h incubation, 20 VOCs were identified, whereby concentrations of 13 VOCs were significantly decreased and of 7 VOCs were significantly increased (p<0.05). The composition of the detected VOCs pattern was different for each cell line and further varied according to incubation period. Ten of the VOCs in the T₂₄, T₄₈ and T₇₂ h incubation period were found to be common to all groups.

In Format-2 setup, including four co-culture same-cell pair combinations, after T₂₄ h incubation period, there was a difference in the peak area of 24 VOCs in all cells compared with the control, whereby concentrations of 23 VOCs were found to be significantly increased and concentration of one VOC (Heptane, 4-methyl-) was found to be significantly decreased (p <0.05) (Table 3). After T₄₈ h incubation period differences in the peak area of 25 VOCs between all cell lines and control medium were detected whereby concentration of 19 VOCs were significantly increased, and of 6 VOCs were significantly decreased (p<0.05). At T₇₂ h incubation, 32 VOCs were identified, whereby concentrations of 5 VOCs were significantly decreased and of 27 VOCs were significantly increased (p<0.05). After T₂₄, T₄₈ and T₇₂ h incubation period, 17 of the VOCs were found to be common to all groups. While not all VOCs found here could be related to endogenous sources, metabolism and signaling, some are. For example, both setups of mono-culture and same cell unconnected co-culture show an increase in cell-metabolism related compounds, such as 3-methyl-3-buten-1-ol (The Human Metabolome Database, HMDB, registry HMDB0030126), that can have both endogenous and exogenous source and relate to cell signaling. This compound may be part of the byproducts of dolichols in the mevalonate pathway (Iannelli. F, et al., 2018) or byproducts of isopentenyl pyrophosphate (IPP) and dimethylallyl pyrophosphate (DMAPP) that can also be hydrolyzed by different Nudix hydrolase enzymes (Srouji. R, et al., 2017) like isopentenyl-diphosphate delta-isomerase 1 (HMDBP01541).

In Format-3 setup, including 6 co-culture different-cell pair combinations (FIG. 1C), after T₂₄ h incubation period, there was a difference in the peak area of 25 VOCs in all the groups as compared with the control, whereby concentrations of 20 VOCs were found to be significantly increased and of 5 VOCs significantly decreased (p<0.05) (Table 4). After T₄₈ h incubation period differences in the peak area of 31 VOCs between all groups and control medium were detected, whereby concentrations of 20 VOCs were significantly increased, and of 11 VOCs were significantly decreased (p<0.05). At T₇₂ h incubation, 37 VOCs were identified, whereby concentrations of 5 VOCs were significantly decreased and of 32 VOCs were significantly increased (p<0.05). These VOCs included mostly ketones and hydrocarbons.

Interestingly, only five VOCs were commonly found at T₂₄, T₄₈ and T₇₂ h incubation in all groups, including 2-methyl butanal, Benzaldehyde, Tetradecane, Acetophenone and Benzophenone. Importantly, the results show that mono-cultured groups bad different volatile profile composition than that of the co-culture combination groups (Tables 2-4). Without wishing to being bound by theory or mechanism of action, the higher number of detected VOCs can be attributed to the combination of the VOC's mixture of these four cell line isolates.

Based on these results, it can be assumed that different small chemicals take part in cell signaling and proliferation. Some of these molecules are also VOCs; as such, they have the potential of triggering changes when sharing the same headspace, implying that such molecules can move between liquid and gaseous phase, and thereby eventually inducing biochemical changes, such as the rate of proliferation (as in FIGS. 3A-3E).

It has therefore been demonstrated that upon communication in co-culture pair combinations (Format-3), VOCs are released from the cells in the conditioned media, which are capable of inducing in-vitro cross-talk between cells. Taken together, the data indicate that cancer cells communicate and that this cross-talk induces changes in the VOCs levels of both different-cell and same-cell pair groups. It has been revealed that the VOCs recorded in the headspace of cells-pair combinations differed from the volatile profile of the respective mono-cultures. Mostly, the VOCs produced by mono-cultures were not detected in cells-pair combinations. In particular, decrease in the hydrocarbons concentration and increase in the ketone concentrations have been shown, corresponding to various signaling pathways.

### Example 5: VOCs Analysis of Co-Culture System Setups Including 2-Cell and 4-Cell Samples-Results

An isolation process was carried out for each experimental format, that enabled reaching Two and Four-cells ahead of time. As the (bio) chemical behavior of the cells changes with time, the VOCs' profile at different time points, including T₀. T₂₄ and T₄₈ h, has also been examined. The VOCs were analyzed using GC-MS, as described in Megjugorac. N. J, et al., Immunol. Invest. 2007, 36, 739.

VOCs obtained from all cell levels and cell level combinations are summarized in Tables 5a-5b. Unexpectedly, cyclohexanone which was produced by the cells at 2-cell level was not detected in the co-culture and mono-culture of bulk samples when cultured under same headspace physically unconnected and connected groups. Similar result was observed for the compounds decane, undecane and tridecane produced by the mono-cultures at 4-cell level but not produced during the interaction of these cell lines. Without wishing to being bound by theory or mechanism of action, it can be contemplated that the results suggest that the production of such potential signaling-VOCs in nature depends strongly on the inter-specific interactions. Most of the VOCs that were tentatively identified within this study (˜50%) are hydrocarbons (Tables 5a-5b). It was further shown that VOCs emitted by the mono-cultures of all cell types at two- and four-cell levels were also found in the bulk cells and could be the cause for the induced changes in bulk cell morphology and cell growth rates observed, as explained hereinabove.

TABLE 1
Co-culture induced apoptosis (%) in Lung cancer cells

| | Groups | | | | | | | |
|---|---|---|---|---|---|---|---|---|
| | Mono- | | Co-culture | | Co-culture | | Co-culture | |
| | culture | | unconnected | | unconnected | | connected | |
| | (Alone) | | Same-cell pair | | different-cell pair | | pair | |
| | Time | | | | | | | |
| | 0 h | 48 h | 0 h | 48 h | 0 h | 48 h | 0 h | 48 h |
| BEAS- | 1.07 | 1.7 | 2.1 | 4.1 | 4.4¹ | 12.8¹ | 4.5¹ | 47.1¹ |
| 2B | | | | | 3.9² | 17.9² | 3.2² | 45.2² |
| | | | | | 4.3³ | 16.2³ | 4.6³ | 42.6³ |
| A549 | 0.7 | 1.1 | 1.2 | 2.42 | 2.4¹ | 3.4¹ | 3.1¹ | 12.7¹ |
| | | | | | 2.7² | 4.3² | 4.1² | 19.3² |
| | | | | | 2.4⁴ | 3.4⁴ | 1.6⁴ | 12.5⁴ |
| H1299 | 0 | 1.04 | 1.74 | 3.01 | 1.0² | 4.1² | 1.8² | 9.7² |
| | | | | | 1.7⁴ | 4.5⁴ | 8.1⁴ | 21.2⁴ |
| | | | | | 2.4³ | 4.3³ | 6.7³ | 54.1³ |
| H1975 | 1.01 | 2.02 | 1.04 | 2.71 | 2.4¹ | 4.6¹ | 0.9¹ | 2.2¹ |
| | | | | | 1.1⁴ | 3.0⁴ | 4.3⁴ | 19.3⁴ |
| | | | | | 2.7³ | 4.3³ | 3.9³ | 19.7³ |

Note:
¹with H1299;
²with H1975;
³with A549;
⁴with BEAS-2B

TABLE 2
List of the VOCs obtained from mono-culture groups (Format-1)
that were increased or decreased relative to the control
normal lung cells at 24, 48 and 72 h of incubation.

| | | | |
|---|---|---|---|
| | | Cate- | p- |
| Compound | CAS No. | gory | value |

At 24 h Incubation

| | | | |
|---|---|---|---|
| 1-Dodecanol | 112-53-8 | ↑* | <0.01 |
| Heptane, 2,2,4,6,6-pentamethyl- | 629-59-4 | ↑* | 0.02 |
| Tetradecane | 629-62-9 | ↑ | 0.03 |
| Nonane, 5-methyl-5-propyl- | 61141-72-8 | ↑ | 0.04 |
| Dodecane, 4,6-dimethyl- | 124-19-6 | ↑* | <0.01 |
| Nonanal | 104-76-7 | ↑ | 0.01 |
| 1-Hexanol, 2-ethyl- | 13475-82-6 | ↑* | <0.01 |
| 2,4-Dimethyl-1-heptane | 19549-87-2 | ↑ | 0.04 |
| 2,4,6-Trimethylpyridine | 108-75-8 | ↑ | 0.02 |
| Undecane, 3,8-dimethyl- | 17301-30-3 | ↑* | <0.01 |
| Butanal, 3-methyl- | 590-86-3 | ↑* | <0.01 |
| 3-Methyl-3-buten-1-ol | 763-32-6 | ↑ | 0.01 |
| 3-Octanol | 589-98-0 | ↑ | 0.01 |
| Acetophenone | 98-86-2 | ↑* | <0.01 |
| Benzaldehyde | 100-52-7 | ↑ | 0.02 |
| Benzophenone | 119-61-9 | ↑* | <0.01 |
| 2,5-Dimethyl-1-heptene | 19549-87-2 | ↓ | 0.04 |
| 2,5-Dimethyl-2,5-hexanediol | 110-03-2 | ↓ | 0.02 |
| 4,6-Dimethyl-2-heptanone | 19549-80-5 | ↓ | 0.05 |
| 2-Methyl-2-hepten-6-one | 110-93-0 | ↑* | <0.01 |
| 3-Methyl-3-buten | 763-32-6 | ↑ | 0.01 |
| 5-Octanol | 589-98-0 | ↑ | 0.01 |
| 1,4-Diacetylbenzene | 1009-61-6 | ↑* | <0.01 |

At 48 h Incubation

| | | | |
|---|---|---|---|
| 1-Dodccanol | 112-53-8 | ↑** | <0.001 |
| 2,2,4-Trimethyl-1,3-pentanediol | 6846-50-0 | ↑ | 0.01 |
| diisobutyrate | | | |
| 2,2-Dimethyloxetane | 6245-99-4 | ↑* | <0.01 |
| 2,4,6-Trimethylpyridine | 108-75-8 | ↑ | 0.01 |
| 2,4-Di-tert-butylphenol | 96-76-4 | ↑ | 0.01 |
| 2-Butanone | 78-93-3 | ↑* | <0.01 |
| 2-Hexanone | 591-78-6 | ↑ | 0.01 |
| 2-Methyl-1-propanol | 78-83-1 | ↑ | 0.01 |
| 2-Octanone | 111-13-7 | ↑ | 0.01 |
| 2-Pentanone | 107-87-9 | ↑* | <0.01 |
| 3-Methyl-3-buten-1-ol | 763-32-6 | ↑ | 0.01 |
| 4-Isopropoxy-2-butanone | 32541-58-5 | ↑ | 0.02 |
| 4-Methylbenzyl alcohol | 589-18-4 | ↑* | <0.01 |
| Acetonylacetone | 110-13-4 | ↑* | <0.01 |
| Acetophenone | 98-86-2 | ↑* | <0.01 |
| Diethyl ether | 60-29-7 | ↑* | <0.01 |
| Dimethyl succinate | 106-65-0 | ↑** | <0.001 |
| Ethanol | 64-17-5 | ↑ | 0.01 |
| Isobutyric acid 2-ethyl-3- | 74367-31-0 | ↑ | 0.01 |
| hydroxyhexyl ester | | | |
| Methyl vinyl ketone | 78-94-4 | ↑* | <0.01 |
| o-Xylene | 95-47-6 | ↓ | 0.05 |
| Pyrrole | 109-97-7 | ↑* | <0.01 |
| trans-4-Methyl-2-pentene | 674-76-0 | ↑ | 0.01 |

At 72 h Incubation

| | | | |
|---|---|---|---|
| 2,6-Di-tert-butylphenol | 128-39-2 | ↑ | 0.01 |
| 3-methyl butanal | 590-86-3 | ↑ | <0.01 |
| 2,4-Dimethyl-1-heptane | 19549-87-2 | ↑ | 0.01 |
| Benzaldehyde | 100-52-7 | ↑ | 0.01 |
| Benzophenone | 119-61-9 | ↑* | <0.01 |
| Dodecane, 4,6-dimethyl- | 124-19-6 | ↑* | <0.01 |
| Cyclohexanone | 108-94-1 | ↓ | 0.02 |
| Phenol | 108-95-2 | ↓ | 0.04 |
| p-Tolualdehyde | 104-87-0 | ↑* | <0.01 |
| 1-Butanol | 71-36-3 | ↓ | 0.05 |
| 2-Methyl-2-hepten-6-one | 110-93-0 | ↑* | <0.01 |
| Acetonitrile | 75-05-8 | ↑* | <0.01 |
| 2,4,6-Trimethylpyridine | 108-75-8 | ↑ | 0.02 |
| Undecane, 3,8-dimethyl- | 17301-30-3 | ↑* | <0.01 |
| Butanal, 3-methyl- | 590-86-3 | ↑* | <0.01 |
| Heptane, 2,2,4,6,6-pentamethyl- | 629-59-4 | ↓ | 0.04 |
| Isophorone | 78-59-1 | ↓ | 0.02 |
| Butylparaben | 94-26-8 | ↑ | 0.01 |
| Tributyl phosphate | 126-73-8 | ↓ | 0.03 |
| Nonane, 5-methyl-5-propyl- | 61141-72-8 | ↓ | 0.02 |

Note:
Significantly different VOCs (p < 0.05) based on averaged peak area (n = 3).
The changes indicate the difference in the averaged peak area between the cells.
(↑: Increased;
↓: decreased;
↑*: <0.01;
↑**: <0.001).

TABLE 3
List of the VOCs obtained from same-cell pair co-culture headspace
groups (Format-2) that were increased or decreased relative to
the control normal lung cells at 24, 48 and 72 h of incubation.

| | | | |
|---|---|---|---|
| | | Cate- | p- |
| Compound | CAS No. | gory | value |

At “24” h

| | | | |
|---|---|---|---|
| 4-Isopropoxy-2-butanone*** | 32541-58-5 | ↑* | 0.02 |
| 2-methyl butanal | 96-17-3 | ↑ | 0.03 |
| Cyclohexanone^{a} | 108-94-1 | ↑* | <0.01 |
| Tridecane | 629-50-5 | ↑ | 0.02 |
| Dimethyl succinate*** | 106-65-0 | ↑* | <0.01 |
| 1-Hexanol, 2-ethyl-*** | 104-76-7 | ↑* | <0.01 |
| Acetophenone*** | 98-86-2 | ↑ | 0.01 |
| Tetradecane*** | 629-59-4 | ↑* | <0.01 |
| 2,4-Di-tert-butylphenol*** | 96-76-4 | ↑ | 0.01 |
| Hexadecane*** | 544-76-3 | ↑* | <0.01 |
| Benzophenone*** | 119-61-9 | ↑* | <0.01 |
| Benzene, 1,3-bis(1,1-dimethylethyl)-*** | 1014-60-4 | ↑ | 0.01 |
| Heptane, 2,2,4,6,6-pentamethyl-*** | 13475-82-6 | ↑* | <0.01 |
| Heptane, 4-methyl-*** | 589-53-7 | ↓ | 0.04 |
| 2,4-Dimethyl-1-heptane*** | 19549-87-2 | ↑ | 0.01 |
| Diethyl ether*** | 60-29-7 | ↑* | <0.01 |
| 1-Dodecanol | 112-53-8 | ↑* | <0.01 |
| Nonane, 5-methyl-5-propyl-*** | 61141-72-8 | ↑ | 0.04 |
| Dodecane, 4,6-dimethyl-*** | 124-19-6 | ↑* | <0.01 |
| Nonanal*** | 104-76-7 | ↑ | 0.01 |
| 2-Methyl-2-hepten-6-one*** | 110-93-0 | ↑* | <0.01 |
| 3-Methyl-3-buten | 763-32-6 | ↑ | 0.01 |
| 5-Octanol | 589-98-0 | ↑ | 0.01 |
| 1,4-Diacetylbenzene | 1009-61-6 | ↑* | <0.01 |

At “48” h

| | | | |
|---|---|---|---|
| Nonanc, 5-methyl-5-propyl-*** | 17312-75-3 | ↑ | 0.04 |
| Dodecane, 4,6-dimethyl-*** | 124-19-6 | ↑* | <0.01 |
| Nonanal*** | 104-76-7 | ↑ | 0.01 |
| 1-Hexanol, 2-ethyl-*** | 13475-82-6 | ↑* | <0.01 |
| 2,4-Dimethyl-1-heptane*** | 19549-87-2 | ↑ | 0.04 |
| 2,4,6-Trimethylpyridine*** | 108-75-8 | ↑ | 0.02 |
| Undecane, 3,8-dimethyl- | 17301-30-3 | ↑* | <0.01 |
| Butanal, 3-methyl- | 590-86-3 | ↑* | <0.01 |
| 3-Methyl-3-buten-1-ol*** | 763-32-6 | ↑ | 0.01 |
| 3-Octanol | 589-98-0 | ↑ | 0.01 |
| Acetophenone*** | 98-86-2 | ↑* | <0.01 |
| Benzaldehyde*** | 100-52-7 | ↑ | 0.02 |
| Benzophenone*** | 119-61-9 | ↑* | <0.01 |
| Heptane, 4-methyl-*** | 589-53-7 | ↓ | 0.05 |
| Benzene, 1,3-bis(1,1-dimethylethyl)-*** | 1014-60-4 | ↓ | 0.02 |
| Dodecane | 112-40-3 | ↓ | 0.03 |
| Pentadecane*** | 629-62-9 | ↑* | <0.01 |
| 4-Isopropoxy-2-butanone*** | 32541-58-5 | ↓ | 0.04 |
| 4-Methylbenzyl alcohol*** | 589-18-4 | ↑* | <0.01 |
| Diethyl ether*** | 60-29-7 | ↓ | 0.05 |
| Dimethyl succinate*** | 106-65-0 | ↑* | <0.01 |
| Undecane | 1120-21-4 | ↑* | <0.01 |
| Cyclohexanone*** | 108-94-1 | ↑* | <0.01 |
| Hexadecane*** | 544-76-3 | ↓ | 0.04 |
| Tetradecane*** | 629-59-4 | ↑ | 0.01 |

At “72” h

| | | | |
|---|---|---|---|
| Diethyl ether*** | 60-29-7 | ↑* | <0.01 |
| Nonane, 5-methyl-5-propyl-*** | 61141-72-8 | ↑ | 0.01 |
| Nonanal*** | 104-76-7 | ↑* | <0.01 |
| Benzaldehyde*** | 100-52-7 | ↑* | <0.01 |
| 2-Pentanone | 107-87-9 | ↑* | <0.01 |
| 3-Methyl-3-buten-1-ol*** | 763-32-6 | ↑ | 0.01 |
| 4-Isopropoxy-2-butanone*** | 32541-58-5 | ↑ | 0.02 |
| 2-Hexanone | 591-78-6 | ↑ | 0.01 |
| Dodecane, 4,6-dimethyl-*** | 124-19-6 | ↑ | 0.01 |
| Cyclohexanone*** | 108-94-1 | ↑ | 0.01 |
| 4-Methylbenzyl alcohol*** | 589-18-4 | ↑* | <0.01 |
| Decane | 124-18-5 | ↑* | <0.01 |
| Tetradecane*** | 629-59-4 | ↑ | 0.02 |
| Pentadecane*** | 629-62-9 | ↑ | 0.01 |
| 2,4-Di-tert-butylphenol*** | 96-76-4 | ↑* | <0.01 |
| 3-methyl-2-Heptene | 3404-75-9 | ↑ | 0.02 |
| 2-Butanone | 78-93-3 | ↑* | <0.01 |
| 3-methyl butanal | 590-86-3 | ↑* | <0.01 |
| 2-Methyl-1-propanol | 78-83-1 | ↑ | 0.01 |
| 2-Octanone | 111-13-7 | ↑ | 0.01 |
| 2,4-Dimethyl-1-heptane*** | 19549-87-2 | ↓ | 0.04 |
| Acetonylacetone | 110-13-4 | ↑* | <0.01 |
| Acetophenone*** | 98-86-2 | ↑* | <0.01 |
| Dimethyl succinate*** | 106-65-0 | ↑** | <0.001 |
| Phenol | 108-95-2 | ↓ | 0.04 |
| p-Tolualdehyde | 104-87-0 | ↑* | <0.01 |
| 1-Butanol | 71-36-3 | ↓ | 0.05 |
| 2-Methyl-2-hepten-6-one*** | 110-93-0 | ↑* | <0.01 |
| Acetonitrile | 75-05-8 | ↑* | <0.01 |
| Heptane, 2,2,4,6,6-pentamethyl-*** | 629-59-4 | ↓ | 0.04 |
| Isophorone | 78-59-1 | ↓ | 0.02 |
| Tributyl phosphate | 126-73-8 | ↓ | 0.03 |

Note:
Significantly different VOCs (p < 0.05) based on averaged peak area (n = 3).
The changes indicate the difference in the averaged peak area between the cells.
(↑: Increased;
↓: decreased;
↑*: <0.01;
↑**: <0.001).
***VOCs, which were common to all co-cultured pairs in same-cell pair combinations and which levels were significantly different from the mono-cultures and direct contact co-culture

TABLE 4
List of the VOCs obtained from different-cell pair co-culture headspace
groups (Format-3) that were increased or decreased relative to the
control normal lung cells at 24, 48 and 72 h of incubation.

| | | | |
|---|---|---|---|
| | | Cate- | p- |
| Compound | CAS No. | gory | value |

At “24” h

| | | | |
|---|---|---|---|
| Acetamide, 2-fluoro-*** | 640-19-7 | ↑ | 0.01 |
| Acetic acid, dimethoxy-, methyl ester | 89-91-8 | ↑** | <0.001 |
| 3-Ethyl-3-hexene | 16789-51-8 | ↑ | 0.01 |
| 1-Hexanol, 2-ethyl- | 104-76-7 | ↑* | <0.01 |
| Phenol | 108-95-2 | ↓ | 0.04 |
| Decane, 3,3,5-trimethyl- | 62338-13-0 | ↑ | 0.01 |
| Tetradecane | 629-59-4 | ↑* | <0.01 |
| 2,4-Di-tert-butylphenol | 96-76-4 | ↑ | 0.01 |
| Butylparaben | 94-26-8 | ↑ | 0.01 |
| Benzoic acid, 4-ethoxy-, ethyl ester | 23676-09-7 | ↑ | 0.01 |
| Hexadecane | 544-76-3 | ↑* | <0.01 |
| p-Tolualdehyde | 104-87-0 | ↑* | <0.01 |
| 2-Pontadecanone | 2345-28-0 | ↓ | 0.04 |
| 3,5-di-tert-Butyl-4- | 1620-98-0 | ↓ | 0.02 |
| hydroxybenzaldehyde | | | |
| 2-Heptadecanone | 2922-51-2 | ↓ | 0.05 |
| 5-Acetylpyrimidine | 10325-70-9 | ↑* | <0.01 |
| Ethanol*** | 64-17-5 | ↑ | 0.01 |
| Fumaronitrile*** | 764-42-1 | ↑ | 0.01 |
| 3-Heptene, 3-methyl- | 7300-03-0 | ↑* | <0.01 |
| 3-Hexen-1-ol, propanoate, (Z)- | 33467-74-2 | ↑ | 0.04 |
| Ethanone, 2-(formyloxy)-1-phenyl- | 55153-12-3 | ↑* | <0.01 |
| Benzene, | 1014-60-4 | ↑ | 0.01 |
| 1,3-bis(1,1-dimethylethyl)-*** | | | |
| Octane, 3,5-dimethyl- | 15869-93-9 | ↑* | <0.01 |
| 1,3-Benzodioxole-2-carboxylic acid, | 831-45-8 | ↑ | 0.04 |
| ethyl ester*** | | | |
| Undecane, 4,7-dimethyl- | 17301-32-5 | ↑ | 0.02 |

At “48” h

| | | | |
|---|---|---|---|
| Tripropyl orthoformate | 621-76-1 | ↑* | <0.01 |
| Sulfur dioxide | 7446-09-5 | ↑* | <0.01 |
| Pentanal, 2-methyl- | 123-15-9 | ↑ | 0.01 |
| Neopentane | 463-82-1 | ↑ | 0.01 |
| 2-Heptene, 3-methyl- | 3404-75-9 | ↓ | 0.04 |
| Cyclohexanol | 108-93-0 | ↑* | <0.01 |
| Pentane, 2,2,3,4-tetramethyl- | 1186-53-4 | ↑ | 0.05 |
| 1-Hexanone, 5-methyl-1-phenyl- | 25552-17-4 | ↑* | <0.01 |
| 3-Methylpentan-3-yl propyl carbonate | 1000372-82-3 | ↑* | <0.01 |
| Hexane, 3,3-dimethyl- | 563-16-6 | ↓ | 0.04 |
| Decane, 2,4-dimethyl- | 2801-84-5 | ↓ | 0.02 |
| 2-Heptene, 4-methyl-, (E)- | 66225-17-0 | ↓ | 0.03 |
| Benzene, | 1014-60-4 | ↓ | 0.02 |
| 1,3-bis(1,1-dimethylethyl)-*** | | | |
| 1,3-benzenedicarboxylic acid, | 1000400-59-0 | ↓ | 0.05 |
| 5-(dimethylamino)-*** | | | |
| 4-Fluoro-3-trifluoromethylbenzoic | 1000338-91-4 | ↑* | <0.01 |
| acid, propyl ester | | | |
| Butane, 1-(2-propenyloxy)- | 3739-64-8 | ↑* | <0.01 |
| Benzene | 71-43-2 | ↓ | 0.04 |
| 2-Ethyl-1-hexanol | 1000411-44-8 | ↓ | 0.02 |
| Dodecane | 112-40-3 | ↓ | 0.03 |
| Pentadecane | 629-62-9 | ↑* | <0.01 |
| Benzene, 1,1′-(1,2- | 7694-30-6 | ↑ | 0.01 |
| cyclobutanediyl)bis-, cis- | | | |
| Formaldehyde | 50-00-0 | ↑ | 0.01 |
| 4-Isopropoxy-2-butanone | 32541-58-5 | ↓ | 0.04 |
| 1,9-Dihydropyrene | 28862-02-4 | ↑* | <0.01 |
| Dimethyl succinate | 106-65-0 | ↑* | <0.01 |
| Ethyne, fluoro- | 2713-09-9 | ↑ | 0.01 |
| Acetic acid, hydroxy- | 79-14-1 | ↑* | <0.01 |
| Dimethyl ether | 115-10-6 | ↑ | 0.02 |
| Undecane, 3,6-dimethyl- | 17301-28-9 | ↑* | <0.01 |
| Phenol, 2,5-bis(1,1-dimethylethyl)- | 5875-45-6 | ↑* | <0.01 |
| 4-Methylbenzyl alcohol | 589-18-4 | ↑* | <0.01 |

At “72” h

| | | | |
|---|---|---|---|
| Butanedioic acid, phenyl- | 635-51-8 | ↑* | <0.01 |
| 5,6-Decanedione | 5579-73-7 | ↑* | <0.01 |
| meso-2,5-Dimethyl-3,4-hexanediol | 32388-93-5 | ↑ | 0.02 |
| Hexadecane, 2-methyl- | 1560-92-5 | ↑* | <0.01 |
| Phenol, 2-(4-methyl-6-phenyl-2- | 65644-27-1 | ↑* | <0.01 |
| pyrimidinyl)- | | | |
| Benzoic acid, 2-ethylhexyl ester | 5444-75-7 | ↑ | 0.01 |
| 1-Propene, 3-propoxy- | 1471-03-0 | ↑ | 0.01 |
| Ethyl-1-propenyl ether | 928-55-2 | ↑* | <0.01 |
| Pentane, 3,3-dimethyl- | 562-49-2 | ↑ | 0.02 |
| Propanc, 2-methoxy-2-methyl- | 1634-04-4 | ↑* | <0.01 |
| Decane, 2,5,9-trimethyl- | 62108-22-9 | ↓ | 0.04 |
| Dodecane, 2,7,10-trimethyl- | 74645-98-0 | ↓ | 0.02 |
| Dodecane, 2,6,10-trimethyl- | 3891-98-3 | ↑ | 0.01 |
| 4-Methylbenzylidene-4-methylaniline | 16979-20-7 | ↑ | <0.01 |
| Azetidine, 3-methyl-3-phenyl- | 5961-33-1 | ↑ | 0.01 |
| o-Xylene | 95-47-6 | ↑ | 0.01 |
| 1,3-Benzodioxole-2-carboxylic acid, | 674-76-0 | ↑* | <0.01 |
| ethyl ester*** | | | |
| Isobutyric acid 2-ethyl-3- | 74367-31-0 | ↓ | 0.05 |
| hydroxyhexyl ester | | | |
| Ethanol*** | 64-17-5 | ↑* | <0.01 |
| 4,6-Dimethyl-2-heptanone | 19549-80-5 | ↑ | 0.01 |
| 2,2-Dimethyloxetane | 6245-99-4 | ↑ | 0.01 |
| Methyl vinyl ketone | 78-94-4 | ↑ | 0.01 |
| Methyltartronic acid | 595-98-2 | ↑* | <0.01 |
| Acetamide, 2-fluoro-*** | 640-19-7 | ↑* | <0.01 |
| .alpha.-Aminoisobutanoic acid | 62-57-7 | ↓ | 0.02 |
| L-Alanine, methyl ester | 10065-72-2 | ↑ | 0.04 |
| Fumaronitrile*** | 764-42-1 | ↑ | 0.02 |
| 1,3-benzenedicarboxylic acid, | 1000400-59-0 | ↑* | <0.01 |
| 5-(dimethylamino)-*** | | | |
| Pyrrole | 109-97-7 | ↑ | 0.01 |
| trans-4-Methyl-2-pentene | 674-76-0 | ↑* | <0.01 |
| 2,6-Di-tert-butylphenol | 128-39-2 | ↑ | 0.02 |
| Cyclobutanecarboxylic acid, | 1000282-21-7 | ↑* | <0.01 |
| 3-methylbutyl ester | | | |
| 3,4-Dimethoxycinnamic acid | 2316-26-9 | ↓ | 0.04 |
| Dodecyl octyl ether | 1000406-38-4 | ↑ | 0.01 |
| Hexestrol | 84-16-2 | ↑ | 0.01 |
| Butanimidamide | 107-90-4 | ↑ | <0.01 |
| N-Methyltaurine | 107-68-6 | ↑ | 0.01 |

Note:
Significantly different VOCs (p < 0.05) based on averaged peak area (n = 3).
The changes indicate the difference in the averaged peak area between the cells.
(↑: Increased;
↓: decreased;
↑*: <0.01;
↑**: <0.001).
***VOCs, which were common to all co-cultured pairs in different-cell pair combinations and which levels were significantly different from the mono-cultures and direct contact co-culture

TABLE 5a
List of the VOCs obtained from 1-cell, 2-cell and 4-cell level experiments

| | | 1-Cell level | | | | 2-Cell level | |
|---|---|---|---|---|---|---|---|
| VOCs | Coding | A | B | C | D | A | B |
| 2-methyl butanal | VOC1 | 0.091953 | 0.231986 | 0.011851 | 0.228483 | 0.002 | 0.205 |
| Benzaldehyde | VOC2 | 0.047123 | 0.012561 | 0.002277 | 0.208454 | 5E−04 | 0.013 |
| 3-methyl butanal | VOC3 | 0.106485 | 0.012541 | 0.002481 | 0.193598 | 6E−04 | 0.014 |
| Pentadecanal- | VOC4 | 0 | 0 | 0 | 0.920081 | 0 | 0 |
| Nonanal | VOC5 | 0 | 0.053533 | 0.066392 | 0.035473 | 0 | 0.05 |
| Dodecane | VOC6 | 0.018508 | 0 | 0 | 0.312 | 0.002 | 0 |
| Tetradecane | VOC7 | 0.139648 | 0.013292 | 0.004482 | 0.285456 | 0.004 | 0.009 |
| Hexadecane | VOC8 | 0 | 0 | 0.298975 | 0.26175 | 0 | 0 |
| Pentadecane | VOC9 | 0.086801 | 0 | 0.003304 | 0.04872 | 0 | 0 |
| Undecane, 3,8-dimethyl- | VOC10 | 0 | 0 | 1.03317 | 0.104456 | 0 | 0 |
| Dodecane, 4,6-dimethyl- | VOC11 | 0.105414 | 0 | 0 | 0.244652 | 0 | 0 |
| Nonane, 4,5-dimethyl- | VOC12 | 0 | 0 | 0 | 0.338502 | 0.001 | 0 |
| Tridecane | VOC13 | 0 | 0 | 0 | 0.146209 | 0.002 | 0 |
| Decane | VOC14 | 0 | 0.011568 | 0 | 0.195015 | 0 | 0 |
| Undecane | VOC15 | 0 | 0.010992 | 0 | 0.717254 | 0 | 0.076 |
| Acetophenone | VOC16 | 0.031025 | 0.016178 | 0.01988 | 0.969789 | 3E−04 | 0.337 |
| Cyclohexanone | VOC17 | 0 | 0.008737 | 0 | 0.781046 | 0 | 0.205 |
| Benzophenone | VOC18 | 0.001284 | 0.024227 | 0.159048 | 0.689972 | 0.004 | 4E−0 |
| Hexadecanoic acid, butyl ester | VOC19 | 0 | 0 | 0 | 0.201954 | 0 | 1.757 |
| 1-Hexanol, 2-ethyl- | VOC20 | 0.025012 | 0.014022 | 0.312705 | 0.233677 | 0.118 | 0.96 |
| 2,4-Di-text-butylphenol | VOC21 | 0.02877 | 0.025295 | 0.005265 | 0.306143 | 0 | 0 |
| 3-methyl-2-Heptene | VOC22 | 0 | 0.018803 | 0 | 0.924003 | 0.001 | 0 |
| 2,4-Dimethyl-1-heptane | VOC23 | 0.02151 | 0.005567 | 0.005 | 1.129222 | 0 | 0 |
| Nonane, 5-methyl-5-propyl- | VOC24 | 0.130682 | 0 | 0 | 0.921955 | 0 | 0 |
| Heptane, 2,2,4,6,6-pentamethyl- | VOC25 | 0 | 0.158875 | 0 | 0 | 0 | 0 |
| Heptane, 4-methyl- | VOC26 | 0 | 0 | 0 | 1.407805 | 0 | 0 |
| Acetamide, 2-fluoro- | VOC27 | 0 | 0 | 0 | 0.925116 | 0 | 0 |
| Acetic acid, dimethoxy-, methyl ester | VOC28 | 0 | 0 | 0 | 1.143028 | 0 | 0 |
| 3-Ethyl-3-hexene | VOC29 | 0 | 0 | 0 | 1.589492 | 0 | 0 |
| Phenol | VOC30 | 0 | 0 | 0 | 0.520325 | 0 | 0 |
| Decane, 3,3,5-trimethyl- | VOC31 | 0 | 0 | 0 | 0.783887 | 0 | 0 |
| Benzoic acid, 4-ethoxy-, ethyl ester | VOC32 | 0 | 0 | 0 | 1.032988 | 0 | 0 |
| 2-Pentadecanone | VOC33 | 0 | 0 | 0 | 0.746929 | 0 | 0 |
| 3,5-di-tert-Butyl-4-hydroxybenzaldehyde | VOC34 | 0 | 0 | 0 | 0.909712 | 0 | 0 |
| 2-Heptadecanone | VOC35 | 0 | 0 | 0 | 1.021465 | 0 | 0 |
| Fumaronitrile | VOC36 | 0 | 0 | 0 | 0.823742 | 0 | 0 |
| 3-Heptene, 3-methyl- | VOC37 | 0 | 0 | 0 | 1.05211 | 0 | 0 |
| 3-Hexen-1-ol, propanoate, (Z)- | VOC38 | 0 | 0 | 0 | 1.009107 | 0 | 0 |
| Ethanone, 2-(formyloxy)-1-phenyl- | VOC39 | 0 | 0 | 0 | 2.163467 | 0 | 0 |
| Benzene, 1,3-bis(1,1-dimethylethyl)- | VOC40 | 0 | 0 | 0 | 1.143429 | 0 | 0 |
| Octane, 3,5-dimethyl- | VOC41 | 0 | 0 | 0 | 1.026228 | 0 | 0 |
| 1,3-Benzodioxole-2-carboxylic acid, ethyl ester | VOC42 | 0 | 0 | 0 | 1.020484 | 0 | 0 |
| Undecane, 4,7-dimethyl- | VOC43 | 0 | 0 | 0 | 1.031678 | 0 | 0 |
| Tripropyl orthoformate | VOC44 | 0 | 0 | 0 | 1.076102 | 0 | 0 |
| Pentanal, 2-methyl- | VOC45 | 0 | 0 | 0 | 0.719337 | 0 | 0 |
| Cyclohexanol | VOC46 | 0 | 0 | 0 | 1.250627 | 0 | 0 |
| Pentane, 2,2,3,4-tetramethyl- | VOC47 | 0 | 0 | 0 | 1.012075 | 0 | 0 |
| 1-Hexanone, 5-methyl-1-phenyl- | VOC48 | 0 | 0 | 0 | 2.012136 | 0 | 0 |
| Hexane, 3,3-dimethyl- | VOC49 | 0 | 0 | 0 | 1.057954 | 0 | 0 |
| Decane, 2,4-dimethyl- | VOC50 | 0 | 0 | 0 | 1.037684 | 0 | 0 |
| 2-Heptene, 4-methyl-, (E)- | VOC51 | 0 | 0 | 0 | 0.93388 | 0 | 0 |
| 1,3-benzenedicarboxylic acid, 5-(dimethylamino)- | VOC52 | 0 | 0 | 0 | 2.003597 | 0 | 0 |
| Butane, 1-(2-propenyloxy)- | VOC53 | 0 | 0 | 0 | 1.053045 | 0 | 0 |
| Benzene | VOC54 | 0 | 0 | 0 | 1.085127 | 0 | 0 |
| 2-Ethyl-1-hexanol | VOC55 | 0 | 0 | 0 | 2.002325 | 0 | 0 |
| 4-Isopropoxy-2-butanone | VOC56 | 0 | 0 | 0 | 2.329353 | 0 | 0 |
| Ethyne, fluoro- | VOC57 | 0 | 0 | 0 | 0.228486 | 0 | 0 |
| Acetic acid, hydroxy- | VOC58 | 0 | 0 | 0 | 1.262476 | 0 | 0 |
| Dimethyl ether | VOC59 | 0 | 0 | 0 | 1.043431 | 0 | 0 |
| Undecane, 3,6-dimethyl- | VOC60 | 0 | 0 | 0 | 2.249493 | 0 | 0 |
| Phenol, 2,5-bis(1,1-dimethylethyl)- | VOC61 | 0 | 0 | 0 | 1.376698 | 0 | 0 |
| 4-Methylbenzyl alcohol | VOC62 | 0 | 0 | 0 | 1.024566 | 0 | 0 |
| 5,6-Decanedione | VOC63 | 0 | 0 | 0 | 0.916022 | 0 | 0 |
| Hexadecane, 2-methyl- | VOC64 | 0 | 0 | 0 | 2.002268 | 0 | 0 |
| Benzoic acid, 2-ethylhexyl ester | VOC65 | 0 | 0 | 0 | 2.802885 | 0 | 0 |
| 1-Propene, 3-propoxy- | VOC66 | 0 | 0 | 0 | 1.569098 | 0 | 0 |
| Ethyl-1-propenyl ether | VOC67 | 0 | 0 | 0 | 1.229786 | 0 | 0 |
| Pentane, 3,3-dimethyl- | VOC68 | 0 | 0 | 0 | 1.006421 | 0 | 0 |
| Decane, 2,5,9-trimethyl- | VOC69 | 0 | 0 | 0 | 0.809506 | 0 | 0 |
| Dodecane, 2,7,10-trimethyl- | VOC70 | 0 | 0 | 0 | 1.041229 | 0 | 0 |
| Dodecane, 2,6,10-trimethyl- | VOC71 | 0 | 0 | 0 | 1.326483 | 0 | 0 |
| 4-Methylbenzylidene-4-methylaniline | VOC72 | 0 | 0 | 0 | 1.002513 | 0 | 0 |
| Azetidine, 3-methyl-3-phenyl- | VOC73 | 0 | 0 | 0 | 1.00551 | 0 | 0 |
| Isobutyric acid 2-ethyl-3-hydroxyhexyl ester | VOC74 | 0 | 0 | 0 | 0.888723 | 0 | 0 |
| 4,6-Dimethyl-2-heptanone | VOC75 | 0 | 0 | 0 | 1.008337 | 0 | 0 |
| 2.2-Dimethyloxetane | VOC76 | 0 | 0 | 0 | 1.422095 | 0 | 0 |
| Methyltartronic acid | VOC77 | 0 | 0 | 0 | 1.21744 | 0 | 0 |
| Ethanol | VOC78 | 0 | 0 | 0 | 0.978207 | 0 | 0 |
| L-Alanine, methyl ester | VOC79 | 0 | 0 | 0 | 1.099979 | 0 | 0 |
| trans-4-Methyl-2-pentene | VOC80 | 0 | 0 | 0 | 0.992413 | 0 | 0 |
| 2,6-Di-tert-butylphenol | VOC81 | 0 | 0 | 0 | 0.83889 | 0 | 0 |
| Cyclobutanecarboxylic acid, 3-methylbutyl ester | VOC82 | 0 | 0 | 0 | 1.001843 | 0 | 0 |
| 3,4-Dimethoxycinnamic acid | VOC83 | 0 | 0 | 0 | 1.725978 | 0 | 0 |
| N-Methyltaurine | VOC84 | 0 | 0 | 0 | 2.003145 | 0 | 0 |
| Diethyl ether | VOC85 | 0 | 0 | 0 | 1.010821 | 0 | 0 |
| 2-Methyl-2-hepten-6-one | VOC86 | 0 | 0 | 0 | 1.002342 | 0 | 0 |
| 3-Methyl-3-buten | VOC87 | 0 | 0 | 0 | 1.157018 | 0 | 0 |
| 5-Octanol | VOC88 | 0 | 0 | 0 | 1.656897 | 0 | 0 |
| 1,4-Diacetylbenzene | VOC89 | 0 | 0 | 0 | 1.536377 | 0 | 0 |
| 2,4,6-Trimethylpyridine | VOC90 | 0 | 0 | 0 | 1.247134 | 0 | 0 |
| 3-Octanol | VOC91 | 0 | 0 | 0 | 0.810483 | 0 | 0 |
| 2-Pentanone | VOC92 | 0 | 0 | 0 | 0.639749 | 0 | 0 |
| 2-Hexanone | VOC93 | 0 | 0 | 0 | 1.900711 | 0 | 0 |
| 2-Butanone | VOC94 | 0 | 0 | 0 | 0.968422 | 0 | 0 |
| 2-Methy 1-1-propanol | VOC95 | 0 | 0 | 0 | 1.275132 | 0 | 0 |
| 2-Octanone | VOC96 | 0 | 0 | 0 | 1.050437 | 0 | 0 |
| Acetonylacetone | VOC97 | 0 | 0 | 0 | 0.714204 | 0 | 0 |
| 1-Butanol | VOC98 | 0 | 0 | 0 | 1.138314 | 0 | 0 |
| 2,5-Dimethyl-1-heptene | VOC99 | 0 | 0 | 0 | 1.53057 | 0 | 0 |
| 2,5-Dimethyl-2,5-hexanediol | VOC100 | 0 | 0 | 0 | 1.321165 | 0 | 0 |
| 2-Fluoro-5-trifluoromethylbenzoic acid, propyl ester | VOC101 | 0 | 0 | 0 | 1.522517 | 0 | 0 |
| Acetaldehyde | VOC102 | 0 | 0 | 0 | 2.526214 | 0 | 0 |
| 2-Heptanone, 7,7-dichloro- | VOC103 | 0 | 0 | 0 | 2.196499 | 0 | 0 |
| 1-Acetyl-4,6,8-trimethylazulene | VOC104 | 0 | 0 | 0 | 1.21618 | 0 | 0 |

| | 2-Cell level | | 4-Cell level | | | | |
|---|---|---|---|---|---|---|---|
| VOCs | C | D | A | B | C | D | RPMI |
| 2-methyl butanal | 0.009 | 0.284 | 0.232 | 0.219 | 0.115 | 0.249 | 0.228483 |
| Benzaldehyde | 0.003 | 0.017 | 0.013 | 0.012 | 0.008 | 0.012 | 0.208454 |
| 3-methyl butanal | 0 | 0.012 | 0.013 | 0.014 | 0 | 0.012 | 0.193598 |
| Pentadecanal- | 0.258 | 0.003 | 0 | 0 | 0.003 | 0 | 0.920081 |
| Nonanal | 0.071 | 0 | 0 | 0.051 | 0.278 | 0 | 0.035473 |
| Dodecane | 0 | 0.032 | 0 | 0 | 0 | 0.034 | 0.312 |
| Tetradecane | 0.007 | 0.011 | 0.013 | 0.014 | 0.003 | 0.011 | 0.285456 |
| Hexadecane | 0.051 | 0.004 | 0 | 0 | 0.003 | 0 | 0.26175 |
| Pentadecane | 0 | 0.025 | 0.029 | 0 | 0 | 0 | 0.04872 |
| Undecane, 3,8-dimethyl- | 0.287 | 0.042 | 0 | 0 | 0 | 0 | 0.104456 |
| Dodecane, 4,6-dimethyl- | 0 | 0.082 | 0.089 | 0 | 0 | 0 | 0.244652 |
| Nonane, 4,5-dimethyl- | 0 | 0.002 | 0 | 0 | 0 | 0 | 0.338502 |
| Tridecane | 0 | 0.011 | 0.013 | 0 | 0 | 0 | 0.146209 |
| Decane | 0 | 0 | 0.012 | 0 | 0 | 0.014 | 0.195015 |
| Undecane | 0 | 0 | 0.011 | 0 | 0 | 0.012 | 0.717254 |
| Acetophenone | 0.018 | 0.017 | 0.016 | 0.016 | 0.031 | 0.016 | 0.969789 |
| Cyclohexanone | 0 | 0 | 0.009 | 0 | 0.31 | 0.009 | 0.781046 |
| Benzophenone | 0.07 | 0.027 | 0.024 | 0.025 | 0.07 | 0.023 | 0.689972 |
| Hexadecanoic acid, butyl ester | 0 | 0 | 0 | 0 | 0.855 | 0.02 | 0.201954 |
| 1-Hexanol, 2-ethyl- | 0.027 | 0.011 | 0.014 | 0.013 | 0.367 | 0.011 | 0.233677 |
| 2,4-Di-text-butylphenol | 0.007 | 0.025 | 0.025 | 0.025 | 0.006 | 0.024 | 0.306143 |
| 3-methyl-2-Heptene | 0.007 | 0.012 | 0 | 0.015 | 0 | 0 | 0.924003 |
| 2,4-Dimethyl-1-heptane | 0 | 0 | 0 | 0 | 0 | 0 | 1.129222 |
| Nonane, 5-methyl-5-propyl- | 0 | 0 | 0 | 0 | 0 | 0 | 0.921955 |
| Heptane, 2,2,4,6,6-pentamethyl- | 0 | 0 | 0 | 0 | 0 | 0 | 0 |
| Heptane, 4-methyl- | 0 | 0 | 0 | 0 | 0 | 0 | 1.407805 |
| Acetamide, 2-fluoro- | 0 | 0 | 0 | 0 | 0 | 0 | 0.925116 |
| Acetic acid, dimethoxy-, methyl ester | 0 | 0 | 0 | 0 | 0 | 0 | 1.143028 |
| 3-Ethyl-3-hexene | 0 | 0 | 0 | 0 | 0 | 0 | 1.589492 |
| Phenol | 0 | 0 | 0 | 0 | 0 | 0 | 0.520325 |
| Decane, 3,3,5-trimethyl- | 0 | 0 | 0 | 0 | 0 | 0 | 0.783887 |
| Benzoic acid, 4-ethoxy-, ethyl ester | 0 | 0 | 0 | 0 | 0 | 0 | 1.032988 |
| 2-Pentadecanone | 0 | 0 | 0 | 0 | 0 | 0 | 0.746929 |
| 3,5-di-tert-Butyl-4-hydroxybenzaldehyde | 0 | 0 | 0 | 0 | 0 | 0 | 0.909712 |
| 2-Heptadecanone | 0 | 0 | 0 | 0 | 0 | 0 | 1.021465 |
| Fumaronitrile | 0 | 0 | 0 | 0 | 0 | 0 | 0.823742 |
| 3-Heptene, 3-methyl- | 0 | 0 | 0 | 0 | 0 | 0 | 1.05211 |
| 3-Hexen-1-ol, propanoate, (Z)- | 0 | 0 | 0 | 0 | 0 | 0 | 1.009107 |
| Ethanone, 2-(formyloxy)-1-phenyl- | 0 | 0 | 0 | 0 | 0 | 0 | 2.163467 |
| Benzene, 1,3-bis(1,1-dimethylethyl)- | 0 | 0 | 0 | 0 | 0 | 0 | 1.143429 |
| Octane, 3,5-dimethyl- | 0 | 0 | 0 | 0 | 0 | 0 | 1.026228 |
| 1,3-Benzodioxole-2-carboxylic acid, ethyl ester | 0 | 0 | 0 | 0 | 0 | 0 | 1.020484 |
| Undecane, 4,7-dimethyl- | 0 | 0 | 0 | 0 | 0 | 0 | 1.031678 |
| Tripropyl orthoformate | 0 | 0 | 0 | 0 | 0 | 0 | 1.076102 |
| Pentanal, 2-methyl- | 0 | 0 | 0 | 0 | 0 | 0 | 0.719337 |
| Cyclohexanol | 0 | 0 | 0 | 0 | 0 | 0 | 1.250627 |
| Pentane, 2,2,3,4-tetramethyl- | 0 | 0 | 0 | 0 | 0 | 0 | 1.012075 |
| 1-Hexanone, 5-methyl-1-phenyl- | 0 | 0 | 0 | 0 | 0 | 0 | 2.012136 |
| Hexane, 3,3-dimethyl- | 0 | 0 | 0 | 0 | 0 | 0 | 1.057954 |
| Decane, 2,4-dimethyl- | 0 | 0 | 0 | 0 | 0 | 0 | 1.037684 |
| 2-Heptene, 4-methyl-, (E)- | 0 | 0 | 0 | 0 | 0 | 0 | 0.93388 |
| 1,3-benzenedicarboxylic acid, 5-(dimethylamino)- | 0 | 0 | 0 | 0 | 0 | 0 | 2.003597 |
| Butane, 1-(2-propenyloxy)- | 0 | 0 | 0 | 0 | 0 | 0 | 1.053045 |
| Benzene | 0 | 0 | 0 | 0 | 0 | 0 | 1.085127 |
| 2-Ethyl-1-hexanol | 0 | 0 | 0 | 0 | 0 | 0 | 2.002325 |
| 4-Isopropoxy-2-butanone | 0 | 0 | 0 | 0 | 0 | 0 | 2.329353 |
| Ethyne, fluoro- | 0 | 0 | 0 | 0 | 0 | 0 | 0.228486 |
| Acetic acid, hydroxy- | 0 | 0 | 0 | 0 | 0 | 0 | 1.262476 |
| Dimethyl ether | 0 | 0 | 0 | 0 | 0 | 0 | 1.043431 |
| Undecane, 3,6-dimethyl- | 0 | 0 | 0 | 0 | 0 | 0 | 2.249493 |
| Phenol, 2,5-bis(1,1-dimethylethyl)- | 0 | 0 | 0 | 0 | 0 | 0 | 1.376698 |
| 4-Methylbenzyl alcohol | 0 | 0 | 0 | 0 | 0 | 0 | 1.024566 |
| 5,6-Decanedione | 0 | 0 | 0 | 0 | 0 | 0 | 0.916022 |
| Hexadecane, 2-methyl- | 0 | 0 | 0 | 0 | 0 | 0 | 2.002268 |
| Benzoic acid, 2-ethylhexyl ester | 0 | 0 | 0 | 0 | 0 | 0 | 2.802885 |
| 1-Propene, 3-propoxy- | 0 | 0 | 0 | 0 | 0 | 0 | 1.569098 |
| Ethyl-1-propenyl ether | 0 | 0 | 0 | 0 | 0 | 0 | 1.229786 |
| Pentane, 3,3-dimethyl- | 0 | 0 | 0 | 0 | 0 | 0 | 1.006421 |
| Decane, 2,5,9-trimethyl- | 0 | 0 | 0 | 0 | 0 | 0 | 0.809506 |
| Dodecane, 2,7,10-trimethyl- | 0 | 0 | 0 | 0 | 0 | 0 | 1.041229 |
| Dodecane, 2,6,10-trimethyl- | 0 | 0 | 0 | 0 | 0 | 0 | 1.326483 |
| 4-Methylbenzylidene-4-methylaniline | 0 | 0 | 0 | 0 | 0 | 0 | 1.002513 |
| Azetidine, 3-methyl-3-phenyl- | 0 | 0 | 0 | 0 | 0 | 0 | 1.00551 |
| Isobutyric acid 2-ethyl-3-hydroxyhexyl ester | 0 | 0 | 0 | 0 | 0 | 0 | 0.888723 |
| 4,6-Dimethyl-2-heptanone | 0 | 0 | 0 | 0 | 0 | 0 | 1.008337 |
| 2.2-Dimethyloxetane | 0 | 0 | 0 | 0 | 0 | 0 | 1.422095 |
| Methyltartronic acid | 0 | 0 | 0 | 0 | 0 | 0 | 1.21744 |
| Ethanol | 0 | 0 | 0 | 0 | 0 | 0 | 0.978207 |
| L-Alanine, methyl ester | 0 | 0 | 0 | 0 | 0 | 0 | 1.099979 |
| trans-4-Methyl-2-pentene | 0 | 0 | 0 | 0 | 0 | 0 | 0.992413 |
| 2,6-Di-tert-butylphenol | 0 | 0 | 0 | 0 | 0 | 0 | 0.83889 |
| Cyclobutanecarboxylic acid, 3-methylbutyl ester | 0 | 0 | 0 | 0 | 0 | 0 | 1.001843 |
| 3,4-Dimethoxycinnamic acid | 0 | 0 | 0 | 0 | 0 | 0 | 1.725978 |
| N-Methyltaurine | 0 | 0 | 0 | 0 | 0 | 0 | 2.003145 |
| Diethyl ether | 0 | 0 | 0 | 0 | 0 | 0 | 1.010821 |
| 2-Methyl-2-hepten-6-one | 0 | 0 | 0 | 0 | 0 | 0 | 1.002342 |
| 3-Methyl-3-buten | 0 | 0 | 0 | 0 | 0 | 0 | 1.157018 |
| 5-Octanol | 0 | 0 | 0 | 0 | 0 | 0 | 1.656897 |
| 1,4-Diacetylbenzene | 0 | 0 | 0 | 0 | 0 | 0 | 1.536377 |
| 2,4,6-Trimethylpyridine | 0 | 0 | 0 | 0 | 0 | 0 | 1.247134 |
| 3-Octanol | 0 | 0 | 0 | 0 | 0 | 0 | 0.810483 |
| 2-Pentanone | 0 | 0 | 0 | 0 | 0 | 0 | 0.639749 |
| 2-Hexanone | 0 | 0 | 0 | 0 | 0 | 0 | 1.900711 |
| 2-Butanone | 0 | 0 | 0 | 0 | 0 | 0 | 0.968422 |
| 2-Methy 1-1-propanol | 0 | 0 | 0 | 0 | 0 | 0 | 1.275132 |
| 2-Octanone | 0 | 0 | 0 | 0 | 0 | 0 | 1.050437 |
| Acetonylacetone | 0 | 0 | 0 | 0 | 0 | 0 | 0.714204 |
| 1-Butanol | 0 | 0 | 0 | 0 | 0 | 0 | 1.138314 |
| 2,5-Dimethyl-1-heptene | 0 | 0 | 0 | 0 | 0 | 0 | 1.53057 |
| 2,5-Dimethyl-2,5-hexanediol | 0 | 0 | 0 | 0 | 0 | 0 | 1.321165 |
| 2-Fluoro-5-trifluoromethylbenzoic acid, propyl ester | 0 | 0 | 0 | 0 | 0 | 0 | 1.522517 |
| Acetaldehyde | 0 | 0 | 0 | 0 | 0 | 0 | 2.526214 |
| 2-Heptanone, 7,7-dichloro- | 0 | 0 | 0 | 0 | 0 | 0 | 2.196499 |
| 1-Acetyl-4,6,8-trimethylazulene | 0 | 0 | 0 | 0 | 0 | 0 | 1.21618 |

Note:
A: BEAS-2B; B: A549; C: H1299; D: H1975.

TABLE 5b
List of the VOCs obtained from bulk cells experiments and their combinations

| | | 1 × 10⁴ cell | | | | | | | | | | | | | | |
|---|---|---|---|---|---|---|---|---|---|---|---|---|---|---|---|---|
| VOCs | Coding | A | B | C | D | A + A | A + B | A + C | A + D | B + B | B + C | B + D | C + C | C + D | D + D | RPMI |
| 2-methyl butanal | VOC1 | 0 | 0 | 0 | 0 | 0 | 0 | 0 | 0 | 0 | 0 | 0 | 0 | 0 | 0 | 0.228483 |
| Benzaldehyde | VOC2 | 0 | 0 | 0 | 0 | 3.427459 | 9.290862 | 6.459851 | 6.342892 | 8.160508 | 7.116051 | 3.979886 | 6.050831 | 7.711605 | 6.267285 | 0.208454 |
| 3-methyl butanal | VOC3 | 0 | 0 | 0 | 0 | 7.564253 | 0 | 0 | 0 | 7.711605 | 0 | 0 | 7.570461 | 0 | 5.83436 | 0.193598 |
| Pentadecanal- | VOC4 | 0 | 0 | 0 | 0 | 0 | 0 | 0 | 0 | 0 | 0 | 0 | 0 | 0 | 0 | 0.920081 |
| Nonanal | VOC5 | 0 | 5.353 | 0.0524 | 5.82736 | 0 | 9.910024 | 3.841713 | 0 | 8.790427 | 0 | 7.880053 | 8.570461 | 0 | 0 | 0.035473 |
| Dodecane | VOC6 | 2.93 | | 0 | 7.603689 | 7.231183 | 0 | 0 | 0 | 0 | 0 | 0 | 0 | 0 | 5.611458 | 0.312 |
| Tetradecane | VOC7 | 0.977 | 1.466 | 0 | 7.874452 | 3.119393 | 8.418477 | 9.815148 | 0 | 5.996829 | 6.130953 | 0 | 0 | 7.454034 | 7.786158 | 0.285456 |
| Hexadecane | VOC8 | 0 | 0 | 1.5507 | 9.862867 | 0 | 1 | 0 | 0 | 0 | 0 | 0 | 6.928426 | 0 | 5.29759 | 0.26175 |
| Pentadecane | VOC9 | 3.863 | 0 | 0 | 0 | 10.98249 | 9.675857 | 7.601586 | 6.928096 | 0 | 0 | 0 | 0 | 0 | 7.038018 | 0.04872 |
| Undecane, 3,8-dimethyl- | VOC10 | 0 | 0 | 2.6495 | 0 | 0 | 0 | 7.939188 | 0 | 6.110949 | 0 | 0 | 7.489563 | 0 | 0 | 0.104456 |
| Dodecane, 4,6-dimethyl- | VOC11 | 9.142 | 0 | 0 | 8.847439 | 9.141926 | 2.168644 | 3.956134 | 0 | 0 | 0 | 5.82736 | 0 | 0 | 9.107617 | 0.244652 |
| Nonane, 4,5-dimethyl- | VOC12 | 0 | 0 | 0 | 0 | 0 | 0 | 0 | 0 | 0 | 0 | 0 | 0 | 0 | 0 | 0.338502 |
| Tridecane | VOC13 | 0 | 0 | 0 | 6.48732 | 0 | 0 | 0 | 0 | | 0 | 5.063348 | 0 | 0 | 9.621418 | 0.146209 |
| Decane | VOC14 | 0 | 0 | 0 | 0 | 0 | 7.48278 | 0 | 0 | 6.949867 | 0 | 0 | 0 | 0 | 0 | 0.195015 |
| Undecane | VOC15 | 0 | 1.529 | 0 | 0 | 0 | 4.085696 | 0 | 0 | 9.164531 | 0 | 0 | 0 | 0 | 0 | 0.717254 |
| Acetophenone | VOC16 | 0 | 0 | 0 | 6.624302 | 9.69927 | 7.572689 | 7.245594 | 8.052448 | 0 | 5.82736 | 0 | 0 | 0 | 6.34294 | 0.969789 |
| Cyclohexanone | VOC17 | 0 | 0 | 0 | 8.236215 | 0 | 7.734267 | 0 | 0 | 8.573309 | 8.974719 | 6.286511 | 0 | 0 | 0 | 0.781046 |
| Benzophenone | VOC18 | 0 | 0 | 0 | 9.924131 | 3.954693 | 0 | 0 | 5.81693 | 0 | 0 | 0 | 0 | 10.24014 | 7.349384 | 0.689972 |
| Hexadecanoic acid, butyl | VOC19 | 0 | 0 | 0 | 0 | 0 | 0 | 0 | 0 | 0 | 0 | 0 | 0 | 0 | 0 | 0.201954 |
| ester | | | | | | | | | | | | | | | | |
| 1-Hexanol, 2-ethyl- | VOC20 | 1.669 | 2.134 | 0 | 7.03498 | 4.991552 | 3.741043 | 0 | 0 | 1.509813 | 0 | 0 | 5.863331 | 0 | 6.8732 | 0.233677 |
| 2,4-Di-tert-butylphenol | VOC21 | 4.821 | 0 | 0 | 7.283081 | 9.36431 | 0 | 0 | 0 | 8.542771 | 0 | 0 | 7.756387 | 0 | 9.072323 | 0.306143 |
| 3-methyl-2-Heptene | VOC22 | 0 | 0 | 0 | 0 | 0 | 8.18775 | 0 | 0 | 7.233955 | 2.83436 | 0 | 0 | 0 | 9.84298 | 0.924003 |
| 2,4-Dimethyl-1-heptane | VOC23 | 0 | 1.771 | 0 | 5.248678 | 6.949867 | 4.213762 | 1.823695 | 3.714937 | 9.405144 | 0 | 0 | 0 | 0 | 5.847439 | 1.129222 |
| Nonane, 5-methyl-5-propyl- | VOC24 | 1.728 | 0 | 0 | 8.902306 | 8.974719 | 3.154142 | 4.103895 | 9.877493 | 0 | 0 | 8.236215 | 0 | 2.949989 | 10.48732 | 0.921955 |
| Heptane, 2,2,4,6,6- | VOC25 | 0 | 3.229 | 0 | 7.437288 | 0 | 2.59877 | 0 | 0 | 5.483069 | 3.879043 | 9.241306 | 0 | 2.6295 | 9.531103 | 0 |
| pentamethyl- | | | | | | | | | | | | | | | | |
| Heptane, 4-methyl- | VOC26 | 0 | 0 | 0 | 4.214554 | 0 | 0 | 0 | 0 | 0 | 0 | 6.900803 | 0 | 0 | 6.625114 | 1.407805 |
| Acetamide, 2-fluoro- | VOC27 | 2.736 | 0 | 1.8542 | 0 | 8.1486 | 0 | 0 | 0 | 0 | 0 | 0 | 6.989625 | 0 | 0 | 0.925116 |
| Acetic acid, dimethoxy-, | VOC28 | 2.368 | 0 | 0 | 0 | 10.29028 | 0 | 0 | 2.030417 | 0 | 0 | 0 | 0 | 0 | 0 | 1.143028 |
| methyl ester | | | | | | | | | | | | | | | | |
| 3-Ethyl-3-hexene | VOC29 | 7.01 | 0 | 0 | 0 | 9.580871 | 2.26295 | 1.367445 | 1.080439 | 0 | 0 | 0 | 0 | 0 | 0 | 1.589492 |
| Phenol | VOC30 | 0 | 2.177 | 0 | 3.316754 | 0 | 5.4575 | 0 | 3.797612 | 8.519968 | 7.231183 | 5.247463 | 0 | 3.187493 | 7.128514 | 0.520325 |
| Decane, 3,3,5-trimethyl- | VOC31 | 2.549 | 0 | 1.1004 | 0 | 8.546332 | 4.957162 | 6.854633 | 2.1471 | 0 | 0 | 0 | 5.246484 | 1.905832 | 0 | 0.783887 |
| Benzoic acid, 4-ethoxy-, | VOC32 | 0 | 0 | 7.6306 | 0 | 0 | 0 | 1.898374 | 0 | 0 | 3.411261 | 0 | 9.240903 | 2.638033 | 0 | 1.032988 |
| ethyl ester | | | | | | | | | | | | | | | | |
| 2-Pentadecanone | VOC33 | 4.667 | 0 | 0 | 0 | 9.270313 | 4.026134 | 0 | 7.051238 | 0 | 0 | 0 | 0 | 0 | 0 | 0.746929 |
| 3,5-di-tert-Butyl-4- | VOC34 | 4.548 | 0 | 0 | 4.362438 | 8.374032 | 2.218477 | 2.388792 | 7.374597 | 0 | 0 | 1.819151 | 0 | 0 | 8.932287 | 0.909712 |
| hydroxybenzaldehyde | | | | | | | | | | | | | | | | |
| 2-Heptadecanone | VOC35 | 0 | 6.902 | 0 | 5.21533 | 0 | 4.199578 | 0 | 2.034673 | 10.08871 | 0 | 9.131561 | 0 | 0 | 7.636177 | 1.021465 |
| Fumaronitrile | VOC36 | 0 | 0 | 7.1158 | 10.51736 | 0 | 0 | 0 | 0 | 0 | 0 | 0 | 9.795405 | 4.125989 | 7.327651 | 0.823742 |
| 3-Heptene, 3-methyl- | VOC37 | 2.108 | 0 | 0 | 0 | 10.87945 | 2.322172 | 0 | 0 | 0 | 0 | 0 | 0 | 0 | 0 | 1.05211 |
| 3-Hexen-1-ol, propanoate, | VOC38 | 0 | 1.564 | 6.18 | 5.232471 | 0 | 0 | 6.896421 | 6.933272 | 7.445601 | 0 | 4.68833 | 10.37488 | 7.490709 | 8.681473 | 1.009107 |
| (Z)- | | | | | | | | | | | | | | | | |
| Ethanone, 2-(formyloxy)-1- | VOC39 | 2.851 | 0 | 0 | 0 | 6.619407 | | 2.840973 | 3.771515 | 0 | 0 | 0 | 0 | 0 | 0 | 2.163467 |
| phenyl- | | | | | | | | | | | | | | | | |
| Benzene, 1,3-bis(1,1- | VOC40 | 5.95 | 8.888 | 0 | 3.324165 | 6.93968 | 9.491183 | 0 | 6.241579 | 7.596039 | 3.66258 | 9.582146 | 0 | 0 | 8.246155 | 1.143429 |
| dimethylethyl)- | | | | | | | | | | | | | | | | |
| Octane, 3,5-dimethyl- | VOC41 | 0 | 2.989 | 0 | 2.413907 | 0 | 2.428667 | 0 | 4.767369 | 6.033417 | 0 | 0 | 0 | 3.748822 | 10.29028 | 1.026228 |
| 1,3-Benzodioxole-2- | VOC42 | 2.984 | 1.403 | 1.699 | 0 | 9.293237 | 5.385093 | 4.170092 | 0 | 6.234594 | 5.337599 | 0 | 8.119328 | 0 | 0 | 1.020484 |
| carboxylic acid, ethyl | | | | | | | | | | | | | | | | |
| ester | | | | | | | | | | | | | | | | |
| Undecane, 4,7-dimethyl- | VOC43 | 1.462 | 1.292 | 0 | | 5.498414 | 9.7010258541622 | 0 | 0 | 9.181336 | 0 | 3.90594 | 0 | 0 | 0 | 1.031678 |
| Tripropyl orthoformate | VOC44 | 0 | 4.068 | 0 | 0 | 0 | 3.9578 | 0 | 0 | 9.646733 | 2.1894 | 3.766831 | 0 | 0 | 0 | 1.076102 |
| Pentanal, 2-methyl- | VOC45 | 0 | 2.917 | 0 | 3.563429 | 0 | 0 | 0 | 0 | 6.81241 | 0 | 8.979792 | 0 | 3.893447 | 9.651228 | 0.719337 |
| Cyclohexanol | VOC46 | 0 | 2.39 | 0 | 0 | 0 | 0 | 4.976611 | 0 | 9.910024 | 5.209171 | 1.814657 | 0 | 0 | 0 | 1.250627 |
| Pentane, 2,2,3,4- | VOC47 | 5.014 | 0 | 2.3932 | 0 | 9.28057 | 5.358856 | 7.199803 | 0 | 0 | 5.336139 | 0 | 9.419065 | 3.926332 | 0 | 1.012075 |
| tetramethyl- | | | | | | | | | | | | | | | | |
| 1-Hexanone, 5-methyl-1- | VOC48 | 0 | 7.574 | 0 | 0 | 0 | 5.710058 | 0 | 0 | 8.957702 | 1.043636 | 0.163338 | 0 | 0 | 0 | 2.012136 |
| phenyl- | | | | | | | | | | | | | | | | |
| Hexane, 3,3-dimethyl- | VOC49 | 4.827 | 2.117 | 0 | 0 | 6.03997 | 10.4895 | 2.130928 | 3.06332 | 7.959594 | 1.761153 | 2.403402 | 0 | 0 | 0 | 1.057954 |
| Decane, 2,4-dimethyl- | VOC50 | 5.192 | 0 | 0 | 6.741605 | 9.477446 | 5.9098 | 2.113725 | 3.943696 | 0 | 0 | 2.035833 | 0 | 1.308034 | 9.023638 | 1.037684 |
| 2-Heptene, 4-methyl-, (E)- | VOC51 | 0 | 4.305 | 6.2735 | 7.715152 | 0 | 1.823742 | 3.342078 | 0 | 9.138244 | 0 | 0 | 7.814806 | 0 | 6.986623 | 0.93388 |
| 1,3-benzenedicarboxylic | VOC52 | 4.471 | 0 | 3.7782 | 0 | 8.592315 | 3.328492 | 2.879566 | | 0 | 1.130928 | 0 | 9.571616 | 0 | 0 | 2.003597 |
| acid, 5-(dimethylamino)- | | | | | | | | | | | | | | | | |
| Butane, 1-(2-propenyloxy)- | VOC53 | 7.074 | 0 | 0 | 5.845988 | 9.862867 | 2.505991 | 0 | 0 | 8.181972 | 0 | 0 | 0 | 0.56185 | 9.580919 | 1.053045 |
| Benzene | VOC54 | 0 | 2.326 | 2.326 | 1.377261 | 0 | 4.06688 | 1.81241 | 2.274946 | 7.711377 | 0 | 0 | 7.874452 | 6.241471 | 9.477446 | 1.085127 |
| 2-Ethyl-1-hexanol | VOC55 | 7.062 | 0 | 0 | 0 | 7.299686 | 3.414258 | 2.624302 | 0 | 0 | 0 | 0 | 0 | 0 | 0 | 2.002325 |
| 4-Isopropoxy-2-butanone | VOC56 | 0 | 0 | 0 | 6.659841 | 0 | 0 | 0 | 3.586611 | 0 | 0 | 1.635132 | 0 | 0 | 8.273598 | 2.329353 |
| Ethyne, fluoro- | VOC57 | 0 | 0 | 0 | 7.581027 | 0 | 0 | 0 | 4.010612 | 0 | 0 | 0 | 0 | 1.850476 | 9.972207 | 0.228486 |
| Acetic acid, hydroxy- | VOC58 | 0 | 6.95 | 8.49 | 0 | | 1.770644 | 3.825736 | 0 | 7.178006 | 8.161165 | 0 | 9.956435 | 0 | 0 | 1.262476 |
| Dimethyl ether | VOC59 | 5.451 | 5.822 | 0 | 0 | 9.753882 | 7.869368 | 2.24186 | 0 | 9.696209 | 1.03997 | 0 | 0 | 0 | 0 | 1.043431 |
| Undecane, 3,6-dimethyl- | VOC60 | 0 | | 7.5739 | 5.674909 | 0 | 0 | 3.113068 | 0 | 0 | 2.263246 | 0 | 8.418477 | 7.461836 | 8.974719 | 2.249493 |
| Phenol, 2,5-bis(1,1- | VOC61 | 5.058 | 6.818 | 0 | 0 | 8.643179 | 3.194466 | 4.010612 | 3.336822 | 8.239691 | 0 | 0 | 0 | 0 | 0 | 1.376698 |
| dimethylethyl)- | | | | | | | | | | | | | | | | |
| 4-Methylbenzyl alcohol | VOC62 | 3.841 | 3.718 | 0 | 3.213448 | 7.716392 | 0 | 0 | 3.442437 | 8.46031 | 0 | 1.474866 | 0 | 0 | 9.862867 | 1.024566 |
| 5,6-Decanedione | VOC63 | 0 | 0 | 5.0214 | 0 | 0 | 0 | 3.74427 | 0 | 0 | 1.929086 | 0 | 9.067673 | 0 | 0 | 0.916022 |
| Hexadecane, 2-methyl- | VOC64 | 0 | 0 | 6.3364 | 7.301115 | 0 | 0 | 2.91525 | 0 | 0 | 1.031407 | 0 | 7.510847 | 9.273344 | 8.790427 | 2.002268 |
| Benzoic acid, 2-ethylhexyl | VOC65 | 0 | 0 | 5.1791 | 4.418074 | 0 | 0 | 5.137941 | 2.11191 | 0 | 1.589002 | 0.7462 | 8.513781 | 7.48278 | 8.321741 | 2.802885 |
| ester | | | | | | | | | | | | | | | | |
| 1-Propene, 3-propoxy- | VOC66 | 5.989 | 0 | 10.089 | 0 | 10.1567 | 6.616113 | 4.60147 | 0 | 0 | 1.263787 | 0.991584 | 8.17502 | 0 | 0 | 1.569098 |
| Ethyl-1-propenyl ether | VOC67 | 5.011 | 0 | 6.8723 | 0 | 9.912684 | 2.155922 | 4.955434 | 0 | 0 | 1.422593 | 0 | 6.182356 | 1.116361 | 0 | 1.229786 |
| Pentane, 3,3-dimethyl- | VOC68 | 0 | 0 | 6.6243 | 0 | 0 | 0 | 2.817502 | 0 | 0 | 1.004148 | 0 | 7.492086 | 1.905091 | 0 | 1.006421 |
| Decane, 2,5,9-trimethyl- | VOC69 | 2.016 | 1.322 | 3.717 | 0 | 5.821786 | 1.041819 | 0 | 0 | 8.920272 | 0 | 0 | 9.199209 | 0 | 0 | 0.809506 |
| Dodecane, 2,7,10- | VOC70 | 0 | 5.451 | 6.1264 | 0 | 0 | 1.598118 | 2.708591 | 0 | 9.767424 | 8.971077 | 0 | 7.03498 | 1.422593 | 0 | 1.041229 |
| trimethyl- | | | | | | | | | | | | | | | | |
| Dodecane, 2,6,10- | VOC71 | 0 | 0 | 0 | 5.484148 | 0 | 0 | 0 | 5.065283 | 0 | 0 | 3.383414 | 0 | 1.026701 | 7.283081 | 1.326483 |
| trimethyl- | | | | | | | | | | | | | | | | |
| 4-Methylbenzylidene-4- | VOC72 | 2.104 | 0 | 4.6374 | 0 | 8.279779 | 4.311051 | 7.299686 | 4.219397 | 0 | 3.205374 | 0 | 8.914273 | 1.891375 | 0 | 1.002513 |
| methylaniline | | | | | | | | | | | | | | | | |
| Azetidine, 3-methyl-3- | VOC73 | 0 | 0 | 8.0569 | 0 | 0 | 0 | 6.872265 | 0 | 0 | 3.684575 | 0 | 9.4466 | 2.032733 | 0 | 1.00551 |
| phenyl- | | | | | | | | | | | | | | | | |
| Isobutyric acid 2-ethyl- | VOC74 | 0 | 5.99 | 0 | 0 | 0 | 3.297972 | 0 | 0 | 11.09657 | 0.93968 | 0 | 0 | 0 | 0 | 0.888723 |
| 3-hydroxyhexyl ester | | | | | | | | | | | | | | | | |
| 4,6-Dimethyl-2-heptanone | VOC75 | 5.927 | 0 | 6.8085 | 0 | 8.534725 | 5.963059 | 7.129238 | 0 | 0 | 4.897623 | 0 | 9.518012 | 2.594757 | 0 | 1.008337 |
| 2.2-Dimethyloxetane | VOC76 | 0 | 0 | 4.9305 | 0 | 0 | 0 | 5.974365 | 0 | 0 | 2.377083 | 0 | 8.119328 | 0 | 0 | 1.422095 |
| Methyltartronic acid | VOC77 | 0 | 0 | 0 | 7.097574 | 0 | 0 | 0 | 6.28162 | 0 | 0 | 5.793372 | 0 | 2.663803 | 9.749181 | 1.21744 |
| Ethanol | VOC78 | 0 | 5.613 | 6.3364 | 0 | 0 | 5.415682 | 5.831639 | 0 | 7.65353 | 2.397795 | 1.360451 | 8.442867 | 0.317683 | 0 | 0.978207 |
| L-Alanine, methyl ester | VOC79 | 0 | 0 | 0 | 8.409726 | 0 | 0 | 0 | 6.25736 | 0 | 0 | 3.830803 | 0 | 0 | 8.253436 | 1.099979 |
| trans-4-Methyl-2-pentene | VOC80 | 0 | 0 | 3.9491 | 0 | 0 | 0 | 3.224444 | 0 | 0 | 0.551936 | 0 | 6.288311 | 0 | 0 | 0.992413 |
| 2,6-Di-tert-butylphenol | VOC81 | 0 | 6.826 | 3.9958 | 0 | 0 | 5.981178 | 3.905811 | 0 | 8.773798 | 3.66096 | 0 | 8.345556 | 1.166856 | 0 | 0.83889 |
| Cyclobutanecarboxylic | VOC82 | 0 | 4.821 | 4.9531 | 0 | 0 | 2.077222 | 4.821339 | 0 | 8.513781 | 9.845988 | 0 | 9.675857 | 2.456678 | 0 | 1.001843 |
| acid, 3-methylbutyl ester | | | | | | | | | | | | | | | | |
| 3,4-Dimethoxycinnamic | VOC83 | 0 | 0 | 0 | 5.179098 | 0 | 0 | 0 | 3.876683 | 0 | 0 | 2.754437 | 0 | 0.29063 | 8.211634 | 1.725978 |
| acid | | | | | | | | | | | | | | | | |
| N-Methyltaurine | VOC84 | 0 | 0 | 5.2536 | 0 | 0 | 0 | 2.318158 | 0 | 0 | 4.42651 | 0 | 7.172224 | 3.166468 | 0 | 2.003145 |
| Diethyl ether | VOC85 | 0 | 4.149 | 0 | 5.734938 | 0 | 3.629063 | 0 | 0 | 8.709158 | 2.30302 | 7.176249 | 0 | 5.431346 | 8.848733 | 1.010821 |
| 2-Methyl-2-hepten-6-one | VOC86 | 0 | 0 | 0 | 4.636654 | 0 | 0 | 0 | 5.903122 | 0 | 0 | 8.078151 | 1 | 2.219397 | 9.67556 | 1.002342 |
| 3-Methyl-3-buten | VOC87 | 0 | 0 | 4.7815 | 4.854408 | 0 | 0 | 6.942898 | 6.777133 | 0 | 4.196767 | 4.222191 | 7.716392 | 2.699797 | 10.37982 | 1.157018 |
| 5-Octanol | VOC88 | 0 | 3.18 | 0 | 0 | 0 | 3.088122 | 0 | 0 | 8.397336 | 1.642767 | 1.099464 | 0 | 0 | 0 | 1.656897 |
| 1,4-Diacetylbenzene | VOC89 | 0 | 0 | 0 | 9.8462 | 0 | 0 | 0 | 4.323472 | 0 | 0 | 7.354315 | | 0 | 8.317683 | 1.536377 |
| 2,4,6-Trimethylpyridine | VOC90 | 0 | 0 | 2.6707 | 5.566115 | 0 | 0 | 0.27367 | 5.642987 | 0 | 2.01423 | 3.994701 | 0 | 1.619013 | 9.436959 | 1.247134 |
| 3-Octanol | VOC91 | 0 | 0 | 0 | 7.743994 | 0 | 0 | 0 | 5.867569 | 0 | 0 | 7.186483 | 8.9578 | 2.360163 | 9.326332 | 0.810483 |
| 2-Pentanone | VOC92 | 0 | 0 | 5.421 | 0 | 0 | 0 | 4.885838 | 0 | 0 | 1.288946 | 0 | 9.4466 | 2.294646 | 0 | 0.639749 |
| 2-Hexanone | VOC93 | 0 | 7.399 | 0 | 8.534725 | 0 | 6.612213 | 0 | 6.874125 | 9.332591 | 3.65353 | 8.675629 | 0 | 1.97846 | 8.025269 | 1.900711 |
| 2-Butanone | VOC94 | 3.925 | 8.126 | 0 | 0 | 6.649454 | 8.48024 | 2.850476 | 1.913202 | 8.714995 | 2.527986 | 2.080032 | 0 | 0 | 0 | 0.968422 |
| 2-Methyl-1-propanol | VOC95 | 4.954 | 0 | 0 | 6.943601 | 3.730431 | 5.493979 | 0.361355 | 0 | 0 | 0 | 7.822058 | 0 | 0.462 | 7.051575 | 1.275132 |
| 2-Octanone | VOC96 | 0 | 0 | 7.2483 | 0 | 0 | 0 | 5.28339 | 0 | 0 | 1.514011 | 0 | 9.440453 | 2.778009 | 0 | 1.050437 |
| Acetonylacetone | VOC97 | 0 | 5.359 | 4.1686 | 0 | 0 | 3.868337 | 0 | 0 | 9.135426 | 6.764432 | 3.462314 | 7.808563 | 0 | 0 | 0.714204 |
| 1-Butanol | VOC98 | 0 | 0 | 0 | 6.103307 | 0 | 0 | 0 | 4.893604 | 0 | 0 | 5.358856 | 0 | 5.866106 | 9.576736 | 1.138314 |
| 2,5-Dimethyl-1-heptene | VOC99 | 5.971 | 8.012 | 0 | 0 | 7.083858 | 8.012331 | 2.548564 | 6.619697 | 9.651228 | 2.046883 | 0 | 0 | 0 | 0 | 1.53057 |
| 2,5-Dimethyl-2,5- | VOC100 | 0 | 7.882 | 7.3418 | 6.548564 | 0 | 3.900236 | 5.456678 | 5.28771 | 9.138244 | 7.283081 | 6.344964 | 9.174819 | 6.370888 | 9.28057 | 1.321165 |
| hexanediol | | | | | | | | | | | | | | | | |
| 2-Fluoro-5- | VOC101 | 0 | 1 | 6.0138 | 0 | 0 | 0 | 2.164034 | 0 | 0 | 2.071284 | 0 | 9.135426 | 2.132893 | 0 | 1.522517 |
| trifluoromethylbenzoic | | | | | | | | | | | | | | | | |
| acid, propyl ester | | | | | | | | | | | | | | | | |
| Acetaldehyde | VOC102 | 7.237 | 0 | 4.0608 | | 8.29849 | 2.115052 | 9.268147 | 3.917404 | 0 | 0 | 0 | 8.957702 | 2.628544 | 0 | 2.526214 |
| 2-Heptanone, 7,7-dichloro- | VOC103 | 4.213 | 0 | 0 | 4.11019 | 7.888781 | 3.917116 | 3.296756 | 8.439637 | 0 | 0 | 0 | 0 | | 9.872123 | 2.196499 |
| 1-Acetyl-4,6,8- | VOC104 | 0 | 4.735 | 6.4451 | 0 | 0 | 1.887442 | 2.721168 | 0 | 8.476222 | 5.011085 | 0 | 7.265227 | 0 | 0 | 1.21618 |
| trimethylazulene | | | | | | | | | | | | | | | | |

Note:
A: BEAS-2B; B: A549; C: H1299; D: H1975.

It is appreciated by persons skilled in the art that the present invention is not limited by what has been particularly shown and described hereinabove. Rather the scope of the present invention includes both combinations and sub-combinations of various features described hereinabove as well as variations and modifications. Therefore, the invention is not to be constructed as restricted to the particularly described embodiments, and the scope and concept of the invention will be more readily understood by references to the claims, which follow.

1. A method of evaluating metastatic behavior, apoptotic behavior, and/or cancer therapy response efficiency, comprising the steps of:
a) providing a first cell culture comprising cancer cells and a second cell culture, wherein there is no physical contact between the first cell culture and the second cell culture;
b) co-culturing the first cell culture and the second cell culture having no physical contact therebetween under a mutual headspace;
c) determining concentrations of VOCs in the mutual headspace;
d) comparing the concentrations of the VOCs in the mutual headspace to the concentrations of VOCs in a control sample;
e) identifying a set of VOCs indicative of cell-to-cell signaling in cancer cells in the mutual headspace, wherein the VOCs have concentrations that are significantly different as compared to the control sample; and
f) evaluating metastatic behavior, apoptotic behavior, and/or cancer therapy response efficiency using the set of VOCs identified in step (e).

2. The method according to claim 1, wherein the control sample is obtained from the group consisting of:
a) a headspace of the first cell culture,
b) a headspace of the second cell culture,
c) a headspace of a second set of the first cell culture and a second set of the second cell culture, wherein there is a physical contact between the second set of the first cell culture and the second set of the second cell culture, and
d) any combination thereof.

3. The method according to claim 1, wherein the second cell culture comprises cells selected from the group consisting of cancer cells, which are identical to the cancer cells of the first cell culture, cancer cells, which are distinct from the cancer cells of the first cell culture, and normal cells.

4. The method according to claim 1, wherein the cancer is lung cancer.

5. The method according to claim 1, wherein the step of determining the concentrations of VOCs in the mutual headspace comprises the use of at least one technique selected from the group consisting of Gas-Chromatography (GC), GC-lined Mass-Spectrometry (GC-MS), Gas-Chromatography-Mass Spectrometry (GC-MS) combined with In-tube Extraction (ITEX), and Proton Transfer Reaction Mass-Spectrometry (PTR-MS).

6. The method according to claim 1, wherein the VOCs in the mutual headspace form a pattern, and wherein the step of comparing the concentrations of the VOCs in the mutual headspace to the concentrations of VOCs in a control sample comprises analyzing the pattern of the VOCs with a pattern recognition analyzer comprising at least one algorithm selected from the group consisting of artificial neural network (ANN) algorithm, support vector machine (SVM), discriminant function analysis (DFA), principal component analysis (PCA), Multilayer perceptron (MLP), generalized regression neural network (GRNN), fuzzy inference system (FIS), self-organizing map (SOM), radial basis function (RBF), genetic algorithm (GA), neuro-fuzzy system (NFS), adaptive resonance theory (ART), partial least squares (PLS), multiple linear regression (MLR), principal component regression (PCR), linear discriminant analysis (LDA), cluster analysis, Fisher linear discriminant analysis (FLDA), Soft independent modeling by class analogy (SIMCA), K-nearest neighbors (KNN), fuzzy logic algorithms, canonical discriminant analysis (CDA) and combinations thereof.

7. The method according to claim 1, wherein the set of VOCs comprises at least one VOC selected from the group consisting of 4-isopropoxy-2-butanone, cyclohexanone, dimethyl succinate, 2-ethyl-1-hexanol, acetophenone, tetradecane, 2,4-di-tert-butylphenol, hexadecane, benzophenone, 1,3-bis(1,1-dimethylethyl)-benzene, 2,2,4,6,6-pentamethyl-heptane, 4-methyl-heptane, 2,4-dimethyl-1-heptane, diethyl ether, 5-methyl-5-propyl-nonane, 4,6-dimethyl-dodecane, nonanal, 2-methyl-2-hepten-6-one, 3-methyl-3-buten-1-ol, benzaldehyde, pentadecane, and 4-methylbenzyl alcohol.

8. The method according to claim 1, wherein the set of VOCs comprises the at least one VOC selected from the group consisting of 4-isopropoxy-2-butanone, cyclohexanone, dimethyl succinate, 1,3-bis(1,1-dimethylethyl)-benzene, diethyl ether, 2-methyl-2-hepten-6-one, 3-methyl-3-buten-1-ol and 4-methylbenzyl alcohol.

9. The method according to claim 1, wherein the step of determining the concentrations of VOCs in the mutual headspace is performed within less than about 1 minute from the formation of a mutual headspace.

10. The method according to claim 1, wherein the step of determining the concentrations of VOCs in the mutual headspace is performed after at least about 24 hours from the formation of a mutual headspace.

11. The method according to claim 1, wherein each one of the first cell culture and the second cell culture comprises at least about 1,000 cells.

12. The method according to claim 1, wherein the first cell culture comprises lung cancer cells, and wherein the cancer comprises a p53 mutation.

13. The method according to claim 12, wherein the p53 mutation is a A549 p53 mutation.

14. The method according to claim 12, wherein the p53 mutation is a H1299 p53 mutation.

15. The method according to claim 12, wherein the p53 mutation is a H1975 p53 mutation.

16. The method according to claim 1, wherein the second cell culture comprises normal cells.

17. The method according to claim 16, wherein the normal cells comprise lung cells.

18. The method according to claim 17, wherein the normal cells are BEAS-2B cells.

19. The method according to claim 1, wherein the second cell culture comprises lung cancer cells.

20. The method according to claim 19, wherein the cancer comprises a p53 mutation selected from the group consisting of a A549 p53 mutation, a H1299 p53 mutation, and a H1975 p53 mutation.
